(19) United States
(12) Patent Application Publication
(10) Pub. No.: US 2014/0323504 A1
(43) Pub. Date: Oct. 30, 2014

(54) Pyrazolo[4,3-c]Pyridine Derivatives As Kinase Inhibitors

(71) Applicant: Cellzome Limited, Brentford, Middlesex (GB)

(72) Inventors: Claudio Dagostin, Cambridgeshire (GB); Nigel Ramsden, Cambridgeshire (GB)

(21) Appl. No.: 14/345,512

(22) PCT Filed: Sep. 20, 2012
(86) PCT No.: PCT/EP2012/068504

§ 371 Date: Mar. 18, 2014

(30) Foreign Application Priority Data
Sep. 20, 2011 (EP) 11182020.5

(51) Int. Cl. C07D 487/04; A61K 31/437; C07D 471/04; A61K 31/519

(52) U.S. Cl. CPC C07D 487/04; A61K 31/519; A61K 31/437; C07D 471/04
USPC 514/262.1; 514/265.1; 514/303; 544/262; 544/280; 546/119

(57) ABSTRACT

The present invention relates to compounds of formula (I)

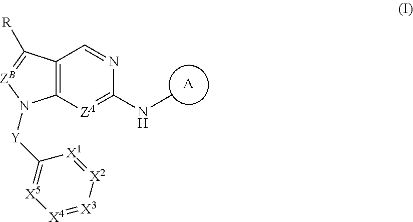

wherein X¹ to X⁵, Y, Z^{A}, Z^{B}, R and A have the meaning as cited in the description and the claims. Said compounds are useful as kinase inhibitors for the treatment or prophylaxis of immunological, inflammatory, autoimmune, allergic disorders, and immunologically-mediated diseases. The invention also relates to pharmaceutical compositions including said compounds as well as the use as medicaments.

The present invention relates to a novel class of kinase inhibitors, including pharmaceutically acceptable salts, which are useful for modulating protein kinase activity for modulating cellular activities such as signal transduction, proliferation, and cytokine secretion. More specifically the invention provides compounds which inhibit kinase activity, in particular JAK3, BTK, BLK, ITK and TEC activity, and signal transduction pathways relating to cellular activities as mentioned above. Furthermore, the present invention relates to pharmaceutical compositions comprising said compounds, for example for the treatment or prevention of an immunological, inflammatory, autoimmune, or allergic disorder or disease or a transplant rejection or a Graft-versus host disease.

Kinases catalyze the phosphorylation of proteins, lipids, sugars, nucleosides and other cellular metabolites and play key roles in all aspects of eukaryotic cell physiology. Especially, protein kinases and lipid kinases participate in the signaling events which control the activation, growth, differentiation and survival of cells in response to extracellular mediators or stimuli such as growth factors, cytokines or chemokines. In general, protein kinases are classified in two groups, those that preferentially phosphorylate tyrosine residues and those that preferentially phosphorylate serine and/or threonine residues. The tyrosine kinases include membrane-spanning growth factor receptors such as the epidermal growth factor receptor (EGFR) and cytosolic non-receptor kinases such as TEC kinases and Janus kinases (JAK).

The TEC family of kinases comprises five members (TEC, BTK, ITK, RLK and BMX) which are expressed mainly by hematopoietic cells and play a central role in signaling through immune receptors such as the high-affinity IgE receptor (FceRI), T cell antigen receptor (TCR) and B cell receptor (BCR). The members of the TEC family share a common protein domain organization. They have an amino-terminal Pleckstrin Homology (PH) domain, a TEC homology domain with one or two proline-rich regions, SRC homology 3 (SH3) and 2 (SH2) protein interaction domains and a carboxy-terminal kinase domain. Activation of the TEC family kinases requires several steps: recruitment to the plasma membrane through their Pleckstrin Homology domain, phosphorylation by SRC family kinases and interactions with proteins that bring them into the vicinity of immune receptor signaling complexes (Schwartzberg et al., 2005, Nature Reviews Immunology 5, 284-295).

TEC family kinases are essential for B cell development and activation. Patients with mutated BTK display a block in B cell development resulting in the almost complete absence of B cells and plasma cells, reduced immunoglobulin levels and an impaired humoral immune response.

In addition, TEC kinases play a role in mast cell activation through the high-affinity IgE receptor (FccRI). ITK and BTK are expressed in mast cells and are activated by FccRI crosslinking. Both acute and late phase inflammatory allergic responses are significantly reduced in ITK-deficient mice when challenged with allergen via the airways. Importantly, airway mast cell degranulation is impaired despite wild-type levels of allergen-specific IgE and IgG1 (Forssell et al., 2005, Am. J. Respir. Cell Mol. Bio. 32, 511-520).

T cells express three TEC kinases (ITK, RLK and TEC) which are activated downstream of the T-cell receptor (TCR) and are involved in TCR signalling. The study of genetically manipulated mice in which the gene encoding the Itk protein is deleted gives important information about the physiological and pathophysiological function of Itk. Itk-deficienct (Itk^{-/-}) mice have specific defects in T helper 2 (T_{H}2) cell responses and reduced pathology in models of allergic asthma. Conversely, ITK expression is increased in T cells from patients with atopic dermatitis, a T_{H}2-cell mediated disease. Therefore ITK has been suggested as a therapeutic target for T_{H}2-cell-mediated diseases (Schwartzberg et al., 2005, Nature Reviews Immunology 5, 284-295).

Another group of kinases that has become a recent focus of drug discovery is the Janus kinase (JAK) family of non-receptor tyrosine kinases. In mammals, the family has four members, JAK1, JAK2, JAK3 and Tyrosine kinase 2 (TYK2). Each protein has a kinase domain and a catalytically inactive pseudo-kinase domain. The JAK proteins bind to cytokine receptors through their amino-terminal FERM (Band-4.1, ezrin, radixin, moesin) domains. After the binding of cytokines to their receptors, JAKs are activated and phosphorylate the receptors, thereby creating docking sites for signalling molecules, especially for members of the signal transducer and activator of transcription (Stat) family (Yamaoka et al., 2004. The Janus kinases (Jaks). Genome Biology 5(12): 253).

In mammals, JAK1, JAK2 and TYK2 are ubiquitously expressed. By contrast, the expression of JAK3 is predominantly in hematopoietic cells and it is highly regulated with cell development and activation (Musso et al., 1995. 181(4):1425-31).

The study of JAK-deficient cell lines and gene-targeted mice has revealed the essential, nonredundant functions of JAKs in cytokine signalling. JAK1 knockout mice display a perinatal lethal phenotype, probably related to the neurological effects that prevent them from sucking (Rodig et al., 1998. Cell 93(3):373-83). Deletion of the JAK2 gene results in embryonic lethality at embryonic day 12.5 as a result of a defect in erythropoiesis (Neubauer et al., 1998. Cell 93(3):397-409). Interestingly, JAK3 deficiency was first identified in humans with autosomal recessive severe combined immunodeficiency (SCID) (Macchi et al., 1995. Nature 377(6544):65-68). JAK3 knockout mice too exhibit SCID but do not display non-immune defects, suggesting that an inhibitor of JAK3 as an immunosuppressant would have restricted effects in vivo and therefore presents a promising drug for immunosuppression (Papageorgiou and Wikman 2004, Trends in Pharmacological Sciences 25(11):558-62).

Activating mutations for JAK3 have been observed in acute megakaryoblastic leukemia (AMKL) patients (Walters et al., 2006. Cancer Cell 10(1):65-75). These mutated forms of JAK3 can transform BaF3 cells to factor-independent growth and induce features of megakaryoblastic leukemia in a mouse model.

Diseases and disorders associated with JAK3 inhibition are further described, for example in WO 01/42246 and WO 2008/060301.

Several JAK3 inhibitors have been reported in the literature which may be useful in the medical field (O'Shea et al., 2004. Nat. Rev. Drug Discov. 3(7):555-64). A potent JAK3 inhibitor (CP-690,550) was reported to show efficacy in an animal model of organ transplantation (Changelian et al., 2003, Science 302(5646):875-888) and clinical trials (reviewed in: Pesu et al., 2008. Immunol. Rev. 223, 132-142). The CP-690,550 inhibitor is not selective for the JAK3 kinase and inhibits JAK2 kinase with almost equipotency (Jiang et al., 2008, J. Med. Chem. 51(24):8012-8018). It is expected that a selective JAK3 inhibitor that inhibits JAK3 with greater potency than JAK2 may have advantageous therapeutic properties, because inhibition of JAK2 can cause anemia (Ghoreschi et al., 2009. Nature Immunol. 4, 356-360).

Pyrimidine derivatives exhibiting JAK3 and JAK2 kinase inhibiting activities are described in WO-A 2008/009458. Pyrimidine compounds in the treatment of conditions in which modulation of the JAK pathway or inhibition of JAK kinases, particularly JAK3 are described in WO-A 2008/118822 and WO-A 2008/118823.

Fluoro substituted pyrimidine compounds as JAK3 inhibitors are described in WO-A 2010/118986. Heterocyclyl pyrazolopyrimidine analogues as JAK inhibitors WO-A 2011/048082.

Pyrrolopyyrimidine compounds are described in WO 2009/098236 A1, WO 2010/100431 A1 and WO 2010/129053 A2.

Pyrazolopyridines and their preparation are known from WO 2006/063820 A1, WO2011/019780 A1 and R. V. Fucini et al., Bioorg. & Med. Chem. Lett. 18 (2008), 5648-5652.

Jak inhibitors are also described in international patent applications with application numbers PCT/EP2012/056887, PCT/EP2012/064510 and PCT/EP2012/064512 as well as WO 2012/022681 A2.

Even though JAK inhibitors are known in the art there is a need for providing additional JAK inhibitors having at least partially more effective pharmaceutically relevant properties, like activity, selectivity especially over JAK2 kinase, and ADME properties.

Thus, an object of the present invention is to provide a new class of kinase inhibitors which may be effective in the treatment or prophylaxis of disorders associated with JAK3, BTK, BLK, ITK or TEC.

Accordingly, the present invention provides compounds of formula (I)

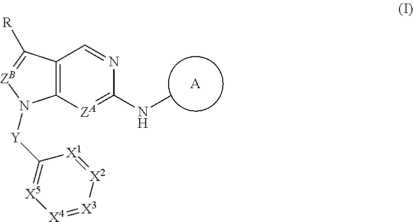

or a pharmaceutically acceptable salt thereof, wherein

### R is H or F;

Z^{A} and Z^{B} are independently selected from the group consisting of CH; and N;
Ring A is phenyl, naphthyl, aromatic 5 to 6 membered heterocyclyl; or aromatic 9 to 11 membered heterobicyclyl, wherein ring A is optionally substituted with one or more R¹;
Each R¹ is independently halogen; CN; C(O)OR²; OR²; C(O)R²; C(O)N(R²R^{2a}); S(O)₂N(R²R^{2a}); S(O)N(R²R^{2a}); S(O)₂R²; S(O)R²; N(R²)S(O)₂N(R^{2a}R^{2b}); N(R)S(O)N(R^{2a}R^{2b}); SR²; N(R²R^{2a}); NO₂; OC(O)R²; N(R²)C(O)R^{2a}; N(R²)S(O)₂R^{2a}; N(R²)S(O)R^{2a}; N(R²)C(O)N(R^{2a}R^{2b}); N(R²)C(O)OR^{2a}; OC(O)N(R²R^{2a}); T¹; C₁₋₆ alkyl; C₂₋₆ alkenyl; or C₂₋₆ alkynyl, wherein C₁ alkyl; C₂₋₆ alkenyl; and C₂₋₆ alkynyl are optionally substituted with one or more R³, which are the same or different;
R², R^{2a}, R^{2b} are independently selected from the group consisting of H; T¹; C₁₋₆ alkyl; C₂₋₆ alkenyl; and C₂₋₆ alkynyl, wherein C₁ alkyl; C₂₋₆ alkenyl; and C₂₋₆ alkynyl are optionally substituted with one or more R³, which are the same or different;
R³ is halogen; CN; C(O)OR⁴; OR⁴; C(O)R⁴; C(O)N(R⁴R^{4a}); S(O)₂N(R⁴R^{4a}); S(O)N(R⁴R^{4a}); S(O)₂R⁴; S(O)R⁴; N(R⁴)S(O)₂N(R^{4a}R^{4b}); N(R⁴)S(O)N(R^{4a}R^{4b}); SR⁴; N(R⁴R^{4a}); NO₂; OC(O)R⁴; N(R⁴)C(O)R^{4a}; N(R⁴)S(O)₂R^{4a}; N(R⁴)S(O)R^{4a}; N(R⁴)C(O)N(R^{4a}R^{4b}); N(R⁴)C(O)OR^{4a}; OC(O)N(R⁴R^{4a}); or T¹;
R⁴, R^{4a}, R^{4b} are independently selected from the group consisting of H; T¹; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more halogen, which are the same or different;
T¹ is C₃₋₇ cycloalkyl; saturated 4 to 7 membered heterocyclyl; or saturated 7 to 11 membered heterobicyclyl, wherein T′ is optionally substituted with one or more R¹⁰, which are the same or different;
Y is (C(R⁵R^{5a}))ₙ;
n is 0, 1, 2, 3 or 4;
R⁵, R^{5a} are independently selected from the group consisting of H; and unsubstituted C₁₋₆ alkyl; or jointly form oxo (═O);
Optionally, R⁵, R^{5a} are joined to form an unsubstituted C₃₋₇ cycloalkyl;
X¹ is C(R⁶) or N; X² is C(R^{6a}) or N; X³ is C(R^{6b}) or N; X⁴ is C(R^{6e}) or N; X⁵ is C(R^{6d}) or N, provided that at most two of X¹, X², X³, X⁴, X⁵ are N;
R⁶, R^{6a}, R^{6b}, R^{6c}, R^{6d} are independently selected from the group consisting of R^{6e}; H; halogen; CN; C(O)OR⁷; OR⁷; C(O)R⁷; C(O)N(R⁷R^{7a}); S(O)₂N(R⁷R^{7a}); S(O)N(R⁷R^{7a}); S(O)₂R⁷; S(O)R⁷; N(R⁷)S(O)₂N(R^{7a}R^{7b}); N(R⁷)S(O)N(R^{7a}R^{7b}); SR⁷; N(R⁷R^{7a}); NO₂; OC(O)R⁷; N(R⁷)C(O)R^{7a}; N(R⁷)S(O)₂R^{7a}; N(R⁷)S(O)R^{7a}; N(R⁷)C(O)N(R^{7a}R^{7b}); N(R⁷)C(O)OR^{7a}; OC(O)N(R⁷R^{7a}); T²; C₁₋₆ alkyl; C₂₋₆ alkenyl; and C₂₋₆ alkynyl, wherein C₁ alkyl; C₂₋₆ alkenyl; and C₂₋₆ alkynyl are optionally substituted with one or more R¹¹, which are the same or different, provided that one of R⁶, R^{6a}, R^{6b}, R^{6c}, R^{6d} is R^{6e}.
R^{6e} is N(R⁷)C(O)C(R^{11a})═C(R^{11b}R^{11c}); N(R⁷)S(O)₂C(R^{11a})═C(R^{11b}R^{11e}); or N(R⁷)C(O)C≡C(R^{11a});

Optionally one of the pairs R⁶/R^{6a}, R^{6a}/R^{6b} is joined to form a ring T³;

R⁷, R^{7a}, R^{7b} are independently selected from the group consisting of H; T²; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more R⁸, which are the same or different;
R⁸ is halogen; CN; C(O)OR⁹; OR⁹; C(O)R⁹; C(O)N(R⁹R^{9a}); S(O)₂N(R⁹R^{9a}); S(O)N(R⁹R^{9a}); S(O)₂R⁹; S(O)R⁹; N(R⁹)S(O)₂N(R^{9a}R^{9b}); N(R⁹)S(O)N(R^{9a}R^{9b}); SR⁹; N(R⁹R^{9a}); NO₂; OC(O)R⁹; N(R⁹)C(O)R^{9a}; N(R⁹)S(O)₂R^{9a}; N(R⁹)S(O)R^{9a}; N(R⁹)C(O)N(R^{9a}R^{9b}); N(R⁹)C(O)OR^{9a}; OC(O)N(R⁹R^{9a}); or T²;
R⁹, R^{9a}, R^{9b} are independently selected from the group consisting of H; T²; C₁₋₆ alkyl; C₂₋₆ alkenyl; and C₂₋₆ alkynyl, wherein C₁₋₆ alkyl; alkenyl; and C₂₋₆ alkynyl are optionally substituted with one or more R¹², which are the same or different;
R¹⁰ is halogen; CN; C(O)OR¹³; OR¹³; oxo (═O), where the ring is at least partially saturated; C(O)R¹³; C(O)N(R¹³R^{13a}); S(O)₂N(R¹³R^{13a}); S(O)N(R¹³R^{13a}); S(O)₂R¹³; S(O)R¹³; N(R¹³)S(O)₂N(R^{13a}R^{13b}); N(R¹³)S(O)N(R^{13a}R^{13b}); SR¹³; N(R¹³R^{13a}); NO₂; OC(O)R¹³); N(R¹³)C(O)R^{13a}; N(R¹³)S(O)₂R^{13a}; N(R¹³)S(O)R^{13a}; N(R¹³)C(O)N(R^{13a}R^{13b}); N(R¹³)C(O)OR^{13a}; OC(O)N(R¹³R^{13a}); C₂₋₆ alkyl; C₂₋₆ alkenyl; or C₂₋₆ alkynyl, wherein C₁₋₆ alkyl; C₂₋₆ alkenyl; and C₂₋₆ alkynyl are optionally substituted with one or more R¹⁴, which are the same or different;
R¹³, R^{13a}, R^{13b} are independently selected from the group consisting of H; C₁₋₅ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more R¹⁴, which are the same or different;

R¹¹, R¹² are independently selected from the group consisting of halogen; CN; C(O)OR¹⁵; OR¹⁵; C(O)R¹⁵; C(O)N(R¹⁵R^{15a}); S(O)₂N(R¹⁵R^{15a}); S(O)N(R¹⁵R^{15a}); S(O)₂R¹⁵; S(O)R¹⁵; N(R¹⁵)S(O)₂N(R^{15a}R^{15b}); N(R¹⁵)S(O)N(R^{15a}R^{15b}); SR¹⁵; N(R¹⁵R^{15a}); NO₂; OC(O)R¹⁵; N(R¹⁵)C(O)R^{15a}; NR¹⁵)S(O)₂R^{15a}; N(R¹⁵)S(O)R^{15a}; N(R¹⁵)C(O)N(R^{15a}R^{15b}); N(R¹⁵)C(O)OR^{15a}; OC(O)N(R¹⁵R^{15a}); and T²;

R^{11a}, R^{11b}, R^{11c} are independently selected from the group consisting of H; halogen; CN; OR¹⁵; C(O)N(R¹⁵R^{15a}); and C₁₋₆ alkyl, wherein C₁₋₆ alkyl is optionally substituted with one or more R¹⁴, which are the same or different;
R¹⁵, R^{15a}, R^{15b} are independently selected from the group consisting of H; T²; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more halogen, which are the same or different;

R¹⁴ is halogen; CN; C(O)OR¹⁶; OR¹⁶; C(O)R¹⁶; C(O)N(R¹⁶R^{16a}); S(O)₂N(R¹⁶R^{16a}); S(O)N(R¹⁶R^{16a}); S(O)₂R¹⁶; S(O)R¹⁶; N(R¹⁶)S(O)₂N(R^{16a}R^{16b}); N(R¹⁶)S(O)N(R^{16a}R^{16b}); SR¹⁶; N(R¹⁶R^{16a}); NO₂; OC(O)R¹⁶; N(R¹⁶)C(O)R^{16a}; N(R¹⁶)S(O)₂R^{16a}; N(R¹⁶)S(O)R^{16a}; N(R¹⁶)C(O)N(R^{16a}R^{16b}); N(R¹⁶)C(O)OR^{16a}; or OC(O)N(R¹⁶R^{16a});

R¹⁶, R^{16a}, R^{16b} are independently selected from the group consisting of H; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more halogen, which are the same or different;
T² is phenyl; naphthyl; indenyl; indanyl; C₃₋₇ cycloalkyl; 4 to 7 membered heterocyclyl; or 7 to 11 membered heterobicyclyl, wherein T² is optionally substituted with one or more R¹⁷, which are the same or different;
T³ is phenyl; C₃₋₇ cycloalkyl; or 4 to 7 membered heterocyclyl, wherein T³ is optionally substituted with one or more R¹⁸, which are the same or different;
R¹⁷, R¹⁸ are independently selected from the group consisting of halogen; CN; C(O)OR¹⁹; OR¹⁹; oxo (═O), where the ring is at least partially saturated; C(O)R¹⁹; C(O)N(R¹⁹R^{19a}); S(O)₂N(R¹⁹R^{19a}); S(O)N(R¹⁹R^{19a}); S(O)₂R¹⁹; S(O)R¹⁹; NR¹⁹)S(O)₂N(R^{19a}R¹⁹); N(R¹⁹)S(O)N(R^{19a}R^{19b}); SR¹⁹; N(R¹⁹R^{19a}); NO₂; OC(O)R¹⁹; N(R¹⁹)C(O)R^{19a}; N(R¹⁹)S(O)₂R^{19a}; N(R¹⁹)S(O)R^{19a}; N(R¹⁹)C(O)N(R^{19a}R^{19b}); N(R¹⁹)C(O)OR^{19a}; OC(O)NR¹⁹R^{19a}); C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more R²⁰, which are the same or different;
R¹⁹, R^{19a}, R^{19b} are independently selected from the group consisting of H; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more R²⁰, which are the same or different;
R²⁰ is halogen; CN; C(O)OR²¹; OR²¹; C(O)R²¹; C(O)NR²¹R^{21a}); S(O)₂N(R²¹R^{21a}); S(O)N(R²¹R^{21a}); S(O)₂R²¹; S(O)R²¹; N(R²¹)S(O)₂N(R^{21a}R^{21b}); N(R²¹)S(O)N(R^{21a}R^{21b}); SR²¹; N(R²¹R^{21a}); NO₂; OC(O)R²¹; N(R²¹)C(O)R^{21a}; N(R²¹)S(O)₂R^{21a}; N(R²¹)S(O)R^{21a}; N(R²¹)C(O)N(R^{21a}R^{21b}); N(R²¹)C(O)OR^{21a}; or OC(O)N(R²¹R^{21a});
R²¹, R^{21a}, R^{21b} are independently selected from the group consisting of H; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more halogen, which are the same or different.

Surprisingly it was found that—without being bound by theory—compounds of the present invention may act as kinase inhibitors that form a covalent bond with their protein target and therefore may have advantageous properties compared to non-covalent inhibitors because they may bind irreversibly to their target protein and inactivate it permanently. After irreversible inhibition of the target, a re-synthesis of the protein may be necessary to restore its function. Therefore, the prolonged duration of the drug action may uncouple the pharmacodynamics of the drug from the pharmacokinetic exposure (Singh et al., 2011. Nat. Rev. Drug Discov. 10(4): 307-317; Singh et al., 2010. Curr. Opin. Chem. Biol. 14(4):475-480).

In case a variable or substituent can be selected from a group of different variants and such variable or substituent occurs more than once the respective variants can be the same or different.

Within the meaning of the present invention the terms are used as follows:

The term “optionally substituted” means unsubstituted or substituted. Generally—but not limited to—, “one or more substituents” means one, two or three, preferably one or two and more preferably one substituents. Generally these substituents can be the same or different.

“Alkyl” means a straight-chain or branched hydrocarbon chain. Each hydrogen of an alkyl carbon may be replaced by a substituent as further specified herein.

“C₁₋₄ alkyl” means an alkyl chain having 1-4 carbon atoms, e.g. if present at the end of a molecule: methyl, ethyl, n-propyl, isopropyl, n-butyl, isobutyl, sec-butyl, tert-butyl, or e.g. —CH₂—, —CH₂—CH₂—, —CH(CH₃)—, —CH₂—CH₂—CH₂—, —CH(C₂H₅)—, —C(CH₃)₂—, when two moieties of a molecule are linked by the alkyl group. Each hydrogen of a C₁₋₄ alkyl carbon may be replaced by a substituent as further specified herein.

“C₁₋₆ alkyl” means an alkyl chain having 1-6 carbon atoms, e.g. if present at the end of a molecule: C₁₋₄ alkyl, methyl, ethyl, n-propyl, isopropyl, n-butyl, isobutyl, sec-butyl; tert-butyl, n-pentyl, n-hexyl, or e.g. —CH₂—, —CH₂—CH₂—, —CH(CH₃)—, —CH₂—CH₂—CH₂—, —CH(C₂H₅)—, —C(CH₃)₂—, when two moieties of a molecule are linked by the alkyl group. Each hydrogen of a C₁₋₆ alkyl carbon may be replaced by a substituent as further specified herein.

“C₃₋₇ cycloalkyl” or “C₃₋₇ cycloalkyl ring” means a cyclic alkyl chain having 3-7 carbon atoms, e.g. cyclopropyl, cyclobutyl, cyclopentyl, cyclohexyl, cyclohexenyl, cycloheptyl. Preferably, cyloalkyl refers to cyclopropyl, cyclobutyl, cyclopentyl, cyclohexyl, or cycloheptyl. Each hydrogen of a cycloalkyl carbon may be replaced by a substituent as further specified herein. The term “C₃₋₅ cycloalkyl” or “C₃₋₅ cycloalkyl ring” is defined accordingly.

“Halogen” means fluoro, chloro, bromo or iodo. It is generally preferred that halogen is fluoro or chloro.

“4 to 7 membered heterocyclyl” or “4 to 7 membered heterocycle” means a ring with 4, 5, 6 or 7 ring atoms that may contain up to the maximum number of double bonds (aromatic or non-aromatic ring which is fully, partially or un-saturated) wherein at least one ring atom up to 4 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for a 4 to 7 membered heterocycles are azetidine, oxetane, thietane, furan, thiophene, pyrrole, pyrroline, imidazole, imidazo line, pyrazole, pyrazoline, oxazole, oxazoline, isoxazole, isoxazoline, thiazole, thiazoline, isothiazole, isothiazoline, thiadiazole, thiadiazoline, tetrahydrofuran, tetrahydrothiophene, pyrrolidine, imidazolidine, pyrazolidine, oxazolidine, isoxazolidine, thiazolidine, isothiazolidine, thiadiazolidine, sulfo lane, pyran, dihydropyran, tetrahydropyran; imidazolidine, pyridine, pyridazine, pyrazine, pyrimidine, piperazine, piperidine, morpholine, tetrazole, triazole, triazolidine, tetrazolidine, diazepane, azepine or homopiperazine. The term “5 to 6 membered heterocyclyl” or “5 to 6 membered heterocycle” is defined accordingly.

“Saturated 4 to 7 membered heterocyclyl” or “saturated 4 to 7 membered heterocycle” means fully saturated “4 to 7 membered heterocyclyl” or “4 to 7 membered heterocycle”.

“Aromatic 5 to 6 membered heterocyclyl” or “aromatic 5 to 6 membered heterocycle” means a heterocycle derived from cyclopentadienyl or benzene, where at least one carbon atom is replaced by a heteroatom selected from the group consisting of sulfur (including —S(O)—, —S(O)₂—), oxygen and nitrogen (including ═N(O)—). Examples for such heterocycles are furan, thiophene, pyrrole, imidazole, pyrazole, oxazole, isoxazole, thiazole, isothiazole, thiadiazole, triazole, tetrazole, pyridine, pyrimidine, pyridazine, pyrazine, triazine.

“Aromatic 5 membered heterocyclyl” or “aromatic 5 membered heterocycle” means a heterocycle derived from cyclopentadienyl, where at least one carbon atom is replaced by a heteroatom selected from the group consisting of sulfur (including —S(O)—, —S(O)₂—), oxygen and nitrogen (including ═N(O)—). Examples for such heterocycles are furan, thiophene, pyrrole, imidazole, pyrazole, oxazole, isoxazole, thiazole, isothiazole, thiadiazole, triazole, tetrazole.

“7 to 11 membered heterobicyclyl” or “7 to 11 membered heterobicycle” means a heterocyclic system of two rings with 7 to 11 ring atoms, where at least one ring atom is shared by both rings and that may contain up to the maximum number of double bonds (aromatic or non-aromatic ring which is fully, partially or un-saturated) wherein at least one ring atom up to 6 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for a 7 to 11 membered heterobicycle are indole, indoline, benzofuran, benzothiophene, benzoxazole, benzisoxazole, benzothiazole, benzisothiazole, benzimidazole, benzimidazoline, quinoline, quinazoline, dihydroquinazoline, quinoline, dihydroquinoline, tetrahydroquinoline, decahydroquinoline, isoquinoline, decahydroisoquino line, tetrahydroisoquinoline, dihydroisoquinoline, benzazepine, purine or pteridine. The term 7 to 11 membered heterobicycle also includes Spiro structures of two rings like 6-oxa-2-azaspiro[3,4]octane, 2-oxa-6-azaspiro[3.3]heptan-6-yl or 2,6-diazaspiro[3.3]heptan-6-yl or bridged heterocycles like 8-aza-bicyclo[3.2.1]octane or 2,5-diazabicyclo[2.2.2]octan-2-yl or 3,8-diazabicyclo[3.2.1]octane.

“Saturated 7 to 11 membered heterobicyclyl” or “saturated 7 to 11 membered heterobicycle” means fully saturated 7 to 11 membered heterobicyclyl or 7 to 11 membered heterobicycle.

“Aromatic 9 to 11 membered heterobicyclyl” or “aromatic 9 to 11 membered heterobicycle” means a heterocyclic system of two rings, wherein at least one ring is aromatic and wherein the heterocyclic ring system has 9 to 11 ring atoms, where two ring atoms are shared by both rings and that may contain up to the maximum number of double bonds (fully or partially aromatic) wherein at least one ring atom up to 6 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for an aromatic 9 to 11 membered heterobicycle are indole, indoline, benzofuran, benzothiophene, benzoxazole, benzisoxazole, benzothiazole, benzisothiazole, benzimidazole, benzimidazo line, quinoline, quinazoline, dihydroquinazoline, dihydroquinoline, tetrahydroquinoline, isoquinoline, tetrahydroisoquinoline, dihydroisoquinoline, benzazepine, purine or pteridine.

Preferred compounds of formula (I) are those compounds in which one or more of the residues contained therein have the meanings given below, with all combinations of preferred substituent definitions being a subject of the present invention. With respect to all preferred compounds of the formula (I) the present invention also includes all tautomeric and stereoisomeric forms and mixtures thereof in all ratios, and their pharmaceutically acceptable salts.

In preferred embodiments of the present invention, the substituents mentioned below independently have the following meaning. Hence, one or more of these substituents can have the preferred or more preferred meanings given below.

Preferably, ring A, Z^{A}, Z^{B} in formula (I) are defined to give formula (Ia)

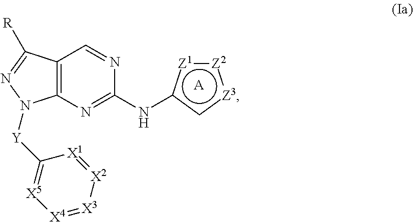

wherein ring A is a aromatic 5 membered heterocycle in which Z¹, Z² and Z³ are independently selected from the group consisting of C(R¹), N, N(R¹), O and S, provided that at least one of Z¹, Z², Z³ is N; and wherein R, Y, X¹ to X⁵ and R¹ are defined above.

Preferably, ring A, Z^{A}, Z^{B}, R in formula (I) are defined to give formula (Ib).

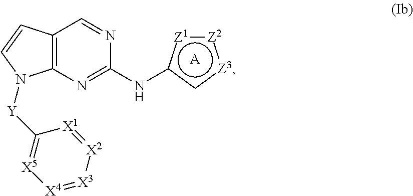

wherein ring A is a membered 5 aromatic heterocycle in which Z¹, Z² and Z³ are independently selected from the group consisting of C(R¹), N, N(R¹), O and S, provided that at least one of Z¹, Z², Z³ is N; and wherein Y, X¹ to X⁵ and R¹ are defined above.
preferably, ring A, Z^{A}, e in formula (I) are defined to give formula (Ic)

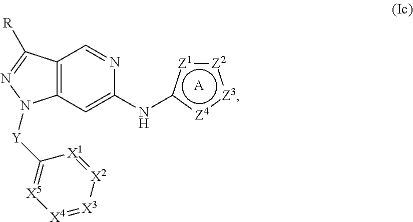

wherein ring A is a aromatic 5 membered heterocycle in which Z¹, Z², Z³ and Z⁴ are independently selected from the group consisting of C(R¹), N, N(R¹), O and S, provided that at least one of Z¹, Z², Z³, Z⁴ is N or N(R¹); and wherein R, Y, X¹ to X⁵ and R¹ are defined above.
preferably, A, Z^{A}, Z^{B} in formula (I) are defined to give formula (Id)

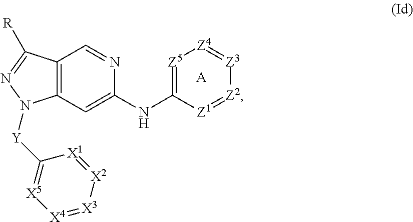

wherein in ring A Z¹ is C(R¹) or N; Z² is C(R¹) or N; Z³ is C(R¹) or N; Z⁴ is C(R¹) or N; Z⁵ is C(R¹), or N, provided that at most two of Z¹, Z², Z³, Z⁴, Z⁵ are N;
optionally two adjacent R¹ are joined to form together with the ring including Z¹ to Z⁵ an aromatic bicyclic ring T⁰;

T⁰ is aromatic 9 to 11 membered heterobicyclyl; naphthyl; indenyl; or indanyl, wherein T⁰ is optionally substituted with one or more R^{1a}, which are the same or different;

R^{1a} is halogen; CN; C(O)OR²; OR²; oxo (═O), where the ring is at least partially saturated; C(O)R²; C(O)N(R²R^{2a}); S(O)₂N(R²R^{2a}); S(O)N(R²R^{2a}); S(O)₂R²; S(O)R²; N(R²)S(O)₂N(R^{2a}R^{2b}); N(R²)S(O)N(R^{2a}R^{2b}); SR²; N(R²R^{2a}); NO₂; OC(O)R²; N(R²)C(O)R^{2a}; N(R²)S(O)₂R^{2a}; N(R²)S(O)R^{2a}; N(R²)C(O)N(R^{2a}R^{2b}); N(R²)C(O)OR^{2a}; OC(O)N(R²R^{2a}); T¹; or C₁₋₆ alkyl, wherein C₁₋₆ alkyl is optionally substituted with one or more R³, which are the same or different; and
wherein R, R¹, R², R^{2a}, R^{2b}, R³, Y, X¹ to X⁵ and R¹ are defined above.

Preferably, R is H.

Preferably, Y is CH₂.

Preferably, none or one (more preferably none) of R⁶, R^{6a}, R^{6b}, R^{6c}, R^{6d} is N.

Preferably, R⁶, R^{6a}, R^{6b}, R^{6c}, R^{6d} are independently selected from the group consisting of R^{6e}; H; halogen; and C₁₋₆ alkyl, wherein C₁₋₆ alkyl is optionally substituted with one or more halogen, which are the same or different, provided that one of R⁶, R^{6a}, R^{6b}, R^{6c}, R^{6d} is R^{6e}. More preferably, one of R⁶, R^{6a}, R^{6b}, R^{6c}, R^{6d} is R^{6e} and at most two (more preferably one, even more preferably none) of the others are other than H.

Preferably, R⁷, R^{11a}, R^{11b}, R^{11c} are independently selected from the group consisting of H; and C₁₋₄ alkyl, wherein C₁₋₄ alkyl is optionally substituted with one or more halogen, which are the same or different.

Preferably, R^{6a} is R^{6e}.

Preferably, R^{6e} is NHC(O)CH═CH₂; NHC(O)C(CH₃)═CH₂; NHC(O)CH═C(CH₃)₂; NHS(O)₂CH═CH₂; or NHC(O)CF—CH.

Preferably, ring A is a pyrazole, an oxazole, an isoxazole, a triazole, a phenyl, or a pyridyl ring. More preferably, ring A is a pyrazolyl ring; even more preferred a ring selected from the group consisting of:

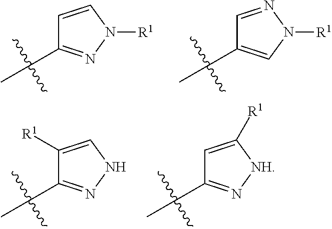

Even more preferred is

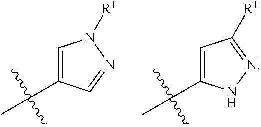

Even more preferred is

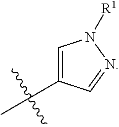

Preferably, 0, 1, or 2 (more preferably 0 or 1, even more preferably 1) R¹, which are the same or different, are other than H.

Preferably, R¹ is C₁₋₄ alkyl, which is optionally substituted with 1 or 2 R³, which are the same or different. Preferably, R¹ is unsubstituted C₁₋₄ alkyl.

Preferably, R³ is halogen; CN; OR⁴; C(O)N(R⁴R^{4a}); or C(O)T¹.

Compounds of formula (I) in which some or all of the above-mentioned groups have the preferred meanings are also an object of the present invention.

Further preferred compounds of the present invention are selected from the group consisting of
N-(3-((6-((1-Methyl-1H-pyrazol-4-yl)amino)-1H-pyrazolo[3,4-d]pyrimidin-1-yl)methyl)phenyl)acrylamide;
N-(2-Fluoro-5-((6-((1-methyl-1H-pyrazol-4-yl)amino)-1H-pyrazolo[3,4-d]pyrimidin-1-yl)methyl)phenyl)acrylamide;
N-(3-((6-((1-Methyl-1H-pyrazol-4-yl)amino)-1H-pyrazolo[3,4-d]pyrimidin-1-yl)methyl)phenyl)propiolamide;
N-(3-((6-((1-Methyl-1H-pyrazol-4-yl)amino)-1H-pyrazolo[3,4-d]pyrimidin-1-yl)methyl)phenyl)ethenesulfonamide;
N-(3-((6-((1-Methyl-1H-pyrazol-4-yl)amino)-1H-pyrazolo[3,4-d]pyrimidin-1-yl)methyl)phenyl)methacrylamide;
3-Methyl-N-(3-((6-((1-methyl-1H-pyrazol-4-yl)amino)-1H-pyrazolo[3,4-d]pyrimidin-1-yl)methyl)phenyl)but-2-enamide;
N-(3-((2-((1-Methyl-1H-pyrazol-4-yl)amino)-7H-pyrrolo[2,3-d]pyrimidin-7-yl)methyl)phenyl)ethenesulfonamide;
N-(3-((6-((1-Methyl-1H-pyrazol-3-yl)amino)-1H-pyrazolo[4,3-c]pyridin-1-yl)methyl)phenyl)acrylamide;
N-(3-((2-((1-Methyl-1H-pyrazol-4-yl)amino)-7H-pyrrolo[2,3-d]pyrimidin-7-yl)methyl)phenyl)acrylamide; and
N-(3-((6-((1-Methyl-1H-pyrazol-4-yl)amino)-1H-pyrazolo[4,3-c]pyridin-1-yl)methyl)phenyl)acrylamide.

Where tautomerism, e.g. keto-enol tautomerism, of compounds of general formula (I) may occur, the individual forms, e.g. the keto and enol form, are comprised separately and together as mixtures in any ratio. The same applies for stereoisomers, e.g. enantiomers, cis/trans isomers, conformers and the like.

Isotopic labeled compounds (“isotopic derivatives”) of formula (I) are also within the scope of the present invention. Methods for isotope labeling are known in the art. Preferred isotopes are those of the elements H, C, N, O and S.

If desired, isomers can be separated by methods well known in the art, e.g. by liquid chromatography. The same applies for enantiomers by using e.g. chiral stationary phases. Additionally, enantiomers may be isolated by converting them into diastereomers, i.e. coupling with an enantiomerically pure auxiliary compound, subsequent separation of the resulting diastereomers and cleavage of the auxiliary residue. Alternatively, any enantiomer of a compound of formula (I) may be obtained from stereoselective synthesis using optically pure starting materials.

The compounds of formula (I) may exist in crystalline or amorphous form. Furthermore, some of the crystalline forms of the compounds of formula (I) may exist as polymorphs, which are included within the scope of the present invention. Polymorphic forms of compounds of formula (I) may be characterized and differentiated using a number of conventional analytical techniques, including, but not limited to, X-ray powder diffraction (XRPD) patterns, infrared (IR) spectra, Raman spectra, differential scanning calorimetry (DSC), thermogravimetric analysis (TGA) and solid state nuclear magnetic resonance (ssNMR).

In case the compounds according to formula (I) contain one or more acidic or basic groups, the invention also comprises their corresponding pharmaceutically or toxicologically acceptable salts, in particular their pharmaceutically utilizable salts. Thus, the compounds of the formula (I) which contain acidic groups can be used according to the invention, for example, as alkali metal salts, alkaline earth metal salts or as ammonium salts. More precise examples of such salts include sodium salts, potassium salts, calcium salts, magnesium salts or salts with ammonia or organic amines such as, for example, ethylamine, ethanolamine, triethanolamine or amino acids. Compounds of the formula (I) which contain one or more basic groups, i.e. groups which can be protonated, can be present and can be used according to the invention in the form of their addition salts with inorganic or organic acids. Examples for suitable acids include hydrogen chloride, hydrogen bromide, phosphoric acid, sulfuric acid, nitric acid, methanesulfonic acid, p-toluenesulfonic acid, naphthalenedisulfonic acids, oxalic acid, acetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, and other acids known to the person skilled in the art. If the compounds of the formula (I) simultaneously contain acidic and basic groups in the molecule, the invention also includes, in addition to the salt forms mentioned, inner salts or betaines (zwitterions). The respective salts according to the formula (I) can be obtained by customary methods which are known to the person skilled in the art like, for example by contacting these with an organic or inorganic acid or base in a solvent or dispersant, or by anion exchange or cation exchange with other salts. The present invention also includes all salts of the compounds of the formula (I) which, owing to low physiological compatibility, are not directly suitable for use in pharmaceuticals but which can be used, for example, as intermediates for chemical reactions or for the preparation of pharmaceutically acceptable salts.

Throughout the invention, the term “pharmaceutically acceptable” means that the corresponding compound, carrier or molecule is suitable for administration to humans. Preferably, this term means approved by a regulatory agency such as the EMEA (Europe) and/or the FDA (US) and/or any other national regulatory agency for use in animals, preferably in humans.

The present invention furthermore includes all solvates of the compounds according to the invention.

According to the present invention “JAK” comprises all members of the JAK family (e.g. JAK1, JAK2, JAK3, and TYK2).

According to the present invention, the expression “JAK1” or “JAK1 kinase” means “Janus kinase 1”.

According to the present invention, the expression “JAK2” or “JAK2 kinase” means “Janus kinase 2”.

According to the present invention, the expression “JAK3” or “JAK3 kinase” means “Janus kinase 3”. The gene encoding JAK3 is located on human chromosome 19p13.1 and it is predominantly in hematopoietic cells. JAK3 is a cytoplasmic protein tyrosine kinase that associates with the gamma-chain of the interleukin 2 (IL-2) receptor. This chain also serves as a component for the receptors of several lymphotropic cytokines, including interleukins IL-4, IL-7, IL-9, IL-15 and IL-21 (Schindler et al., 2007. J. Biol. Chem. 282(28):20059-63). JAK3 plays a key role in the response of immune cells to cytokines, especially in mast cells, lymphocytes and macrophages. Inhibition of JAK3 has shown beneficial effects in the prevention of transplant rejection (Changelian et al., 2003, Science 302(5646):875-888).

Moreover, according to the present invention, the expression “JAK3” or “JAK3 kinase” includes mutant forms of JAK3, preferably JAK3 mutants found in acute megakaryoblastic leukemia (AMKL) patients. More preferred, these mutants are single amino acid mutations. Activating JAK3 mutations were observed in acute megakaryoblastic leukemia (AMKL) patients (Walters et al., 2006. Cancer Cell 10(1):65-75). Therefore, in a preferred embodiment, the expression “JAK” also includes a JAK3 protein having a V7221 or P132T mutation.

According to the present invention, the expression “TYK2” or “TYK2 kinase” means “Protein-Tyrosine kinase 2”.

According to the present invention, the expression “BTK” means “Bruton's tyrosine kinase”.

According to the present invention, the expression “BLK” means “B-lymphocyte specific kinase”.

According to the present invention, the expression “ITK” means “Interleukin-2 (IL-2)-inducible T-cell kinase”.

According to the present invention, the expression “TEC” means “TEC kinase”.

As shown in the examples, compounds of the invention were tested for their potency, selectivity, efficacy and mode of action. Within the JAK family, all tested compound bind JAK3 more potently than JAK1, JAK2 and TYK2 (see Table 6).

Compounds of the invention bind potently to BTK, BLK, ITK and TEC (see Table 8) and inhibit kinase function (see Table 10). In a washout study of cells it Was demonstrated that compounds of the invention that are expected to be covalent inhibitors display long-lasting pharmacological effects for up to four hours whereas two reference inhibitors showed much shorter activity (see Table 12). In addition, it was demonstrated by mass spectrometry analysis that example 1 covalently bound to cystein residue 909 (Cys909) of JAK3 (see Table 15).

Consequently, the compounds of the present invention are considered to be useful for the prevention or treatment of diseases and disorders associated with JAK3, BTK, BLK, ITK or TEC, for example immunological, inflammatory, autoimmune, or allergic disorders, transplant rejection, Graft-versus-Host-Disease or proliferative diseases such as cancer.

In a preferred embodiment, the compounds of the present invention are selective JAK3 inhibitors.

The present invention provides pharmaceutical compositions comprising a compound of formula (I) or a pharmaceutically acceptable salt thereof as active ingredient together with a pharmaceutically acceptable carrier, optionally in combination with one or more other pharmaceutical compositions.

“Pharmaceutical composition” means one or more active ingredients; and one or more inert ingredients that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, including but not limited to peanut oil, soybean oil, mineral oil, sesame oil and the like. Water is a preferred carrier when the pharmaceutical composition is administered orally. Saline and aqueous dextrose are preferred carriers when the pharmaceutical composition is administered intravenously. Saline solutions and aqueous dextrose and glycerol solutions are preferably employed as liquid carriers for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsions, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the therapeutic, preferably in purified form, together with a suitable amount of carrier so as to provide the form for proper administration to the patient. The formulation should suit the mode of administration.

A pharmaceutical composition of the present invention may comprise one or more additional compounds as active ingredients like one or more compounds of formula (I) not being the first compound in the composition or other JAK inhibitors. Further bioactive compounds may be steroids, leukotriene antagonists, cyclosporine or rapamycin.

The compounds of the present invention or pharmaceutically acceptable salt(s) thereof and the other pharmaceutically active agent(s) may be administered together or separately and, when administered separately, this may occur separately or sequentially in any order. When combined in the same formulation it will be appreciated that the two compounds must be stable and compatible with each other and the other components of the formulation. When formulated separately they may be provided in any convenient formulation, conveniently in such manner as are known for such compounds in the art.

It is further included within the present invention that the compound of formula (I), or a pharmaceutically acceptable salt thereof, or a pharmaceutical composition comprising a compound of formula (I) is administered in combination with another drug or pharmaceutically active agent and/or that the pharmaceutical composition of the invention further comprises such a drug or pharmaceutically active agent.

In this context, the term “drug or pharmaceutically active agent” includes a drug or pharmaceutical agent that will elicit the biological or medical response of a tissue, system, animal or human that is being sought, for instance, by a researcher or clinician.

“Combined” or “in combination” or “combination” should be understood as a functional coadministration, wherein some or all compounds may be administered separately, in different formulations, different modes of administration (for example subcutaneous, intravenous or oral) and different times of administration. The individual compounds of such combinations may be administered either sequentially in separate pharmaceutical compositions as well as simultaneously in combined pharmaceutical compositions.

For example, in rheumatoid arthritis therapy, combination with other chemotherapeutic or antibody agents is envisaged. Suitable examples of pharmaceutically active agents which may be employed in combination with the compounds of the present invention and their salts for rheumatoid arthritis therapy include: immunosuppresants such as aintolmctin guacil, mizoribine and rimexolone; anti-TNFα agents such as etanercept, infliximab, Adalimumab, Anakinra, Abatacept, Rituximab; tyrosine kinase inhibitors such as leflunomide; kallikrein antagonists such as subreum; interleukin 11 agonists such as oprelvekin; interferon beta 1 agonists; hyaluronic acid agonists such as NRD-101 (Aventis); interleukin 1 receptor antagonists such as anakinra; CD8 antagonists such as amiprilose hydrochloride; beta amyloid precursor protein antagonists such as reumacon; matrix metalloprotease inhibitors such as cipemastat and other disease modifying anti-rheumatic drugs (DMARDs) such as methotrexate, sulphasalazine, cyclosporin A, hydroxychoroquine, auranofin, aurothioglucose, gold sodium thiomalate and penicillamine.

In particular, the treatment defined herein may be applied as a sole therapy or may involve, in addition to the compounds of the invention, conventional surgery or radiotherapy or chemotherapy. Accordingly, the compounds of the invention can also be used in combination with existing therapeutic agents for the treatment proliferative diseases such as cancer. Suitable agents to be used in combination include:

(i) antiproliferative/antineoplastic drugs and combinations thereof; as used in medical oncology such as alkylating agents (for example cis-platin, carboplatin, cyclophosphamide, nitrogen mustard, Melphalan, chlorambucil, busulphan and nitrosoureas); antimetabolites (for example antifolates such as fluoropyrimidines like 5-fluorouracil and tegafur, raltitrexed, methotrexate, cytosine arabinoside, hydroxyurea and gemcitabine); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like paclitaxel and taxotere); and topoisomerase inhibitors (for example epipodophyllotoxins like etoposide and teniposide, amsacrine, topotecan and camptothecins);
(ii) cytostatic agents such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) anti-invasion agents (for example c-Src kinase family inhibitors like 4-(6-chloro-2,3-methylenedioxyanilino)-7-[2-(4-methylpiperazin-1-yl)ethoxy]-5-tetrahydropyran-4-yloxy-quinazoline (AZD0530) and N-(2-chloro-6-methylphenyl)-2-{6-[4-(2-hydroxyethyppiperazin-1-yl]-2-methylpyrimidin-4-ylamino}thiazole-5-carboxamide (dasatinib, BMS-354825), and metalloproteinase inhibitors like marimastat and inhibitors of urokinase plasminogen activator receptor function);
(iv) inhibitors of growth factor function: for example such inhibitors include growth factor antibodies and growth factor receptor antibodies (for example the anti-erbB2 antibody trastuzumab [Herceptin™] and the anti-erbB1 antibody cetuximab [C225]); such inhibitors also include, for example, tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinaiolin-4-amine (gefitinib, ZD 1839), N-(3-ethynylphenyl)-6,7-bis(2-methoxyethoxy)quinazolin-4-amine (erlotinib, OSI-774) and 6-acrylamido-A-(3-chloro-4-fluorophenyl)-7-(3-morpholinopropoxy)-quinazolin-4-amine (CI-1033) and erbB2 tyrosine kinase inhibitors such as lapatinib), inhibitors of the hepatocyte growth factor family, inhibitors of the platelet-derived growth factor family such as imatinib, inhibitors of serine/threonine kinases (for example RasRaf signalling inhibitors such as farnesyl transferase inhibitors, for example sorafenib (BAY 43-9006)) and inhibitors of cell signalling through MEK and/or Akt kinases;
(v) antiangiogenic agents such as those which inhibit the effects of vascular endothelial growth factor, for example the anti-vascular endothelial cell growth factor antibody bevacizumab (Avastin™) and VEGF receptor tyrosine kinase inhibitors such as 4-(4-bromo-2-fluoroanilino)-6-methoxy-7-(1-methylpiperidin-4-ylmethoxy)quinazoline (ZD6474; Example 2 within WO 01/32651), 4-(4-fluoro-2-methylindol-5-yloxy)-6-methoxy-7-(3-pyrrolidin-1-ylpropoxy)quinazoline (AZD2171; Example 240 within WO 00/47212), vatalanib (PTK787; WO 98/35985) and SU1 1248 (sunitinib; WO 01/60814), and compounds that work by other mechanisms (for example linomide, inhibitors of integrin αvβ3 function and angiostatin);
(vi) vascular damaging agents such as combretastatin A4 and compounds disclosed in International Patent Application WO 99/02166;
(vii) antisense therapies, for example those which are directed to the targets listed above, such, as ISIS 2503, an anti-ras antisense agent;
(viii) gene therapy approaches, including approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and (ix) immunotherapeutic approaches, including ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

Further combination treatments are described in WO-A 2009/008992 and WO-A 2007/107318), incorporated herein by reference.

Accordingly, the individual compounds of such combinations may be administered either sequentially in separate pharmaceutical compositions as well as simultaneously in combined pharmaceutical compositions.

The pharmaceutical compositions of the present invention include compositions suitable for oral, rectal, topical, parenteral (including subcutaneous, intramuscular, and intravenous), ocular (ophthalmic), pulmonary (nasal or buccal inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

In practical use, the compounds of formula (I) can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as powders, hard and soft capsules and tablets, with the solid oral preparations being preferred over the liquid preparations.

Because of their ease of administration, tablets and capsules represent the most advantageous oral dosage unit form in which case solid pharmaceutical carriers are obviously employed. If desired, tablets may be coated by standard aqueous or non-aqueous techniques. Such compositions and preparations should contain at least 0.1 percent of active compound. The percentage of active compound in these compositions may, of course, be varied and may conveniently be between about 2 percent to about 60 percent of the weight of the unit. The amount of active compound in such therapeutically useful compositions is such that an effective dosage will be obtained. The active compounds can also be administered intranasally, for example, as liquid drops or spray.

The tablets, pills, capsules, and the like may also contain a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

Compounds of formula (I) may also be administered parenterally. Solutions or suspensions of these active compounds can be prepared in water suitably mixed with a surfactant such as hydroxypropyl-cellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

Any suitable route of administration may be employed for providing a mammal, especially a human, with an effective dose of a compound of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like. Preferably compounds of formula (I) are administered orally.

The effective dosage of active ingredient employed may vary depending on the particular compound employed, the mode of administration, the condition being treated and the severity of the condition being treated. Such dosage may be ascertained readily by a person skilled in the art.

A therapeutically effective amount of a compound of the present invention will normally depend upon a number of factors including, for example, the age and weight of the animal, the precise condition requiring treatment and its severity, the nature of the formulation, and the route of administration. However, an effective amount of a compound of formula (I) for the treatment of an inflammatory disease, for example rheumatoid arthritis (RA), will generally be in the range of 0.1 to 100 mg/kg body weight of recipient (mammal) per day and more usually in the range of 1 to 10 mg/kg body weight per day. Thus, for a 70 kg adult mammal, the actual amount per day would usually be from 70 to 700 mg and this amount may be given in a single dose per day or more usually in a number (such as two, three, four, five or six) of sub-doses per day such that the total daily dose is the same. An effective amount of a pharmaceutically acceptable salt, prodrug or metabolite thereof, may be determined as a proportion of the effective amount of the compound of formula (I) per se. It is envisaged that similar dosages would be appropriate for treatment of the other conditions referred to above.

As used herein, the term “effective amount” means that amount of a drug or pharmaceutical agent that will elicit the biological or medical response of a tissue, system, animal or human that is being sought, for instance, by a researcher or clinician.

Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

Another aspect of the present invention is a compound of the present invention or a pharmaceutically acceptable salt thereof for use as a medicament.

Another aspect of the present invention is a compound of the present invention or a pharmaceutically acceptable salt thereof for use in a method of treating or preventing a disease or disorder associated with JAK3, BTK, BLK, ITK or TEC.

In the context of the present invention, a disease or disorder associated with JAK3, BTK, BLK, ITK and TEC is defined as a disease or disorder where JAK3, BTK, BLK, ITK or TEC are involved.

In a preferred embodiment, wherein the diseases or disorder is associated with JAK3, BTK, BLK, ITK or TEC is an immunological, inflammatory, autoimmune, or allergic disorder or disease of a transplant rejection or a Graft-versus host disease.

Consequently, another aspect of the present invention is a compound or a pharmaceutically acceptable salt thereof of the present invention for use in a method of treating or preventing an immunological, inflammatory, autoimmune, or allergic disorder or disease of a transplant rejection or a Graft-versus host disease.

Inflammation of tissues and organs occurs in a wide range of disorders and diseases and in certain variations, results from activation of the cytokine family of receptors. Exemplary inflammatory disorders associated with activation of JAK3, BTK, BLK, ITK or TEC include, in a non-limiting manner, skin inflammation due radiation exposure, asthma, allergic inflammation and chronic inflammation.

According to the present invention, an autoimmune disease is a disease which is at least partially provoked by an immune reaction of the body against own components, for example proteins, lipids or DNA. Examples of organ-specific autoimmune disorders are insulin-dependent diabetes (Type I) which affects the pancreas, Hashimoto's thyroiditis and Graves' disease which affect the thyroid gland, pernicious anemia which affects the stomach, Cushing's disease and Addison's disease which affect the adrenal glands, chronic active hepatitis which affects the liver; polycystic ovary syndrome (PCOS), celiac disease, psoriasis, inflammatory bowel disease (1BD) and ankylosing spondylitis. Examples of non-organ-specific autoimmune disorders are rheumatoid arthritis, multiple sclerosis, systemic lupus and myasthenia gravis.

Type I diabetes ensues from the selective aggression of autoreactive T-cells against insulin secreting beta-cells of the islets of Langerhans. Targeting JAK3 in this disease is based on the observation that multiple cytokines that signal through the JAK pathway are known to participate in the T-cell mediated autoimmune destruction of beta-cells. Indeed, a JAK3 inhibitor, JANEX-1 was shown to prevent spontaneous autoimmune diabetes development in the NOD mouse model of type I diabetes.

In a preferred embodiment, the autoimmune disease is selected from the group consisting of rheumatoid arthritis (RA), inflammatory bowel disease (IBD; Crohn's disease and ulcerative colitis), psoriasis, systemic lupus erythematosus (SLE), and multiple sclerosis (MS).

Rheumatoid arthritis (RA) is a chronic progressive, debilitating inflammatory disease that affects approximately 1% of the world's population. RA is a symmetric polyarticular arthritis that primarily affects the small joints of the hands and feet. In addition to inflammation in the synovium, the joint lining, the aggressive front of tissue called pannus invades and destroys local articular structures (Firestein 2003, Nature 423:356-361).

Inflammatory bowel disease (IBD) is characterized by a chronic relapsing intestinal inflammation. IBD is subdivided into Crohn's disease and ulcerative colitis phenotypes. Crohn disease involves most frequently the terminal ileum and colon, is transmural and discontinuous. In contrast, in ulcerative colitis, the inflammation is continuous and limited to rectal and colonic mucosal layers. In approximately 10% of cases confined to the rectum and colon, definitive classification of Crohn's disease or ulcerative colitis cannot be made and are designated ‘indeterminate colitis.’ Both diseases include extraintestinal inflammation of the skin, eyes, or joints. Neutrophil-induced injuries may be prevented by the use of neutrophils migration inhibitors (Asakura et al., 2007, World J. Gastroenterol. 13(15):2145-9).

Psoriasis is a chronic inflammatory dermatosis that affects approximately 2% of the population. It is characterized by red, scaly skin patches that are usually found on the scalp, elbows, and knees, and may be associated with severe arthritis. The lesions are caused by abnormal keratinocyte proliferation and infiltration of inflammatory cells into the dermis and epidermis (Salon et al., 2005, New Engl. J. Med. 352:1899-1912).

Systemic lupus erythematosus (SLE) is a chronic inflammatory disease generated by T cell-mediated B-cell activation, which results in glomerulonephritis and renal failure. Human SLE is characterized at early stages by the expansion of long-lasting autoreactive CD4+ memory cells (D'Cruz et al., 2007, Lancet 369(9561):587-596).

Multiple sclerosis (MS) is an inflammatory and demyelating neurological disease. It has bee considered as an autoimmune disorder mediated by CD4+ type 1 T helper cells, but recent studies indicated a role of other immune cells (Hemmer et al., 2002, Nat. Rev. Neuroscience 3, 291-301).

In a preferred embodiment, the allergic disease is selected from the group consisting of asthma, chronic obstructive pulmonary disease (COPD), adult respiratory distress syndrome (ARDS), bronchitis, conjunctivitis, dermatitis and allergic rhinitis.

Mast cells express JAK3 and JAK3 is a key regulator of the IgE mediated mast cell responses including the release of inflammatory mediators. JAK3 was shown to be a valid target in the treatment of mast cell mediated allergic reaction. Allergic disorders associated with mast cell activation include Type I immediate hypersensitivity reactions such as allergic rhinitis (hay fever), allergic urticaria (hives), angioedema, allergic asthma and anaphylaxis, for example anaphylactic shock. These disorders may be treated or prevented by inhibition of JAK3 activity, for example, by administration of a JAK3 inhibitor according to the present invention.

Transplant rejection (allograft transplant rejection) includes, without limitation, acute and chronic allograft rejection following for example transplantation of kidney, heart, liver, lung, bone marrow, skin and cornea. It is known that T cells play a central role in the specific immune response of allograft rejection. Hyperacute, acute and chronic organ transplant rejection may be treated. Hyperacute rejection occurs within minutes of transplantation. Acute rejection generally occurs within six to twelve months of the transplant. Hyperacute and acute rejections are typically reversible where treated with immunosuppressant agents. Chronic rejection, characterized by gradual loss of organ function, is an ongoing concern for transplant recipients because it can occur anytime after transplantation.

Graft-versus-host disease (GVDH) is a major complication in allogeneic bone marrow transplantation (BMT). GVDH is caused by donor T cells that recognize and react to recipient differences in the histocompatibility complex system, resulting in significant morbidity and mortality. JAK3 plays a key role in the induction of GVHD and treatment with a JAK3 inhibitor, JANEX-1, was shown to attenuate the severity of GVHD (reviewed in Cetkovic-Cvrlje and Ucken, 2004).

Asthma is a complex syndrome with many clinical phenotypes in both adults and children. Its major characteristics include a variable degree of air flow obstruction, bronchial hyperresponsiveness, and airway inflammation (Busse and Lemanske, 2001, N. Engl. J. Med. 344:350-362).

Chronic obstructive pulmonary disease (COPD) is characterized by inflammation, airflow limitation that is not fully reversible, and a gradual loss of lung function. In COPD, chronic inhalation of irritants causes an abnormal inflammatory response, remodeling of the airways, and restriction of airflow in the lungs. The inhaled irritant is usually tobacco smoke, but occupational dust and environmental pollution are variably implicated (Shapiro 2005, N. Engl. J. med. 352, 2016-2019).

In a preferred embodiment, the inflammatory disease is an eye disease.

Dry eye syndrome (DES, also known as keratoconjunctivitis sicca) is one of the most common problems treated by eye physicians. Sometimes DES is referred to as dysfunctional tear syndrome (Jackson, 2009. Canadian Journal Ophthalmology 44(4), 385-394). DES affects up to 10% of the population between the ages of 20 to 45 years, with this percentage increasing with age. Although a wide variety of artificial tear products are available, these products provide only transitory relief of symptoms. As such, there is a need for agents, compositions and therapeutic methods to treat dry eye.

As used herein, “dry eye disorder” is intended to encompass the disease states summarized in a recent official report of the Dry Eye Workshop (DEWS), which defined dry eye as “a multifactorial disease of the tears and ocular surface that results in symptoms of discomfort, visual disturbance, and tear film instability with potential damage to the ocular surface. It is accompanied by increased osmolality of the tear film and inflammation of the ocular surface.”

(Lemp, 2007. “The Definition and Classification of Dry Eye Disease: Report of the Definition and Classification Subcommittee of the International Dry Eye Workshop”, The Ocular Surface, 5(2), 75-92). Dry eye is also sometimes referred to as keratoconjunctivitis sicca. In some embodiments, the treatment of the dry eye disorder involves ameliorating a particular symptom of dry eye disorder, such as eye discomfort, visual disturbance, tear film instability, tear hyperosmolarity, and inflammation of the ocular surface.

Uveitis is the most common form of intraocular inflammation and remains a significant cause of visual loss. Current treatments for uveitis employs systemic medications that have severe side effects and are globally immunosuppressive. Clinically, chronic progressive or relapsing forms of non-infectious uveitis are treated with topical and/or systemic corticosteroids. In addition, macrolides such as cyclosporine and rapamycin are used, and in some cases cytotoxic agents such as cyclophosphamide and chlorambucil, and antimetabolites such as azathioprine, methotrexate, and leflunomide (Srivastava et al., 2010. Uveitis: Mechanisms and recent advances in therapy. Clinica Chimica Acta, doi:10.1016/j.cca.2010.04.017).

Further eye diseases, combination treatments and route of administration are described for example in WO-A 2010/039939, which is hereby incorporated herein by reference.

In a further preferred embodiment, the disease or disorder associated with JAK3, BTK, BLK, ITK or TEC is a proliferative disease, especially cancer.

Diseases and disorders associated especially with JAK3, BTK, BLK, ITK or TEC are proliferative disorders or diseases, especially cancer.

Therefore, another aspect of the present invention is a compound or a pharmaceutically acceptable salt thereof of the present invention for use in a method of treating or preventing a proliferative disease, especially cancer.

Cancer comprises a group of diseases characterized by uncontrolled growth and spread of abnormal cells. All types of cancers generally involve some abnormality in the control of cell growth, division and survival, resulting in the malignant growth of cells. Key factors contributing to said malignant growth of cells are independence from growth signals, insensitivity to anti-growth signals, evasion of apoptosis, limitless replicative potential, sustained angiogenesis, tissue invasion and metastasis, and genome instability (Hanahan and Weinberg, 2000. The Hallmarks of Cancer. Cell 100, 57-70).

Typically, cancers are classified as hematological cancers (for example leukemias and lymphomas) and solid cancers such as sarcomas and carcinomas (for example cancers of the brain, breast, lung, colon, stomach, liver, pancreas, prostate, ovary).

The kinase inhibitors of the present invention may also useful in treating certain malignancies, including skin cancer and hematological malignancy such as lymphomas and leukemias.

Especially cancers in which the JAK-STAT signal transduction pathway is activated, for example due to activation of JAK3 are expected to respond to treatment with JAK3 inhibitors. Examples of cancers harboring JAK3 mutations are acute megakaryoblastic leukemia (AMKL) (Walters et al., 2006. Cancer Cell 10(1):65-75) and breast cancer (Jeong et al., 2008. Clin. Cancer Res. 14, 3716-3721).

Yet another aspect of the present invention is the use of a compound of the present invention or a pharmaceutically acceptable salt thereof for the manufacture of a medicament for the treatment or prophylaxis of diseases and disorders associated with JAK3, BTK, BLK, ITK or TEC.

Yet another aspect of the present invention is the use of a compound of the present invention or a pharmaceutically acceptable salt thereof for the manufacture of a medicament for treating or preventing an immunological, inflammatory, autoimmune, or allergic disorder or disease or a transplant rejection or a Graft-versus host disease.

Yet another aspect of the present invention is the use of a compound of the present invention or a pharmaceutically acceptable salt thereof for the manufacture of a medicament for treating or preventing a proliferative disease, especially cancer.

In the context of these uses of the invention, diseases and disorders associated with JAK3, BTK, BLK, ITK or TEC are as defined above.

Yet another aspect of the present invention is a method for treating, controlling, delaying or preventing in a mammalian patient in need thereof one or more conditions selected from the group consisting of diseases and disorders associated with JAK3, BTK, BLK, ITK or TEC, wherein the method comprises the administration to said patient a therapeutically effective amount of a compound according to present invention or a pharmaceutically acceptable salt thereof.

Yet another aspect of the present invention is a method for treating, controlling, delaying or preventing in a mammalian patient in need thereof one or more conditions selected from the group consisting of an immunological, inflammatory, autoimmune, or allergic disorder or disease or a transplant rejection or a Graft-versus host disease, wherein the method comprises the administration to said patient a therapeutically effective amount of a compound according to present invention or a pharmaceutically acceptable salt thereof.

Yet another aspect of the present invention is a method for treating, controlling, delaying or preventing in a mammalian patient in need thereof a proliferative disease, especially cancer, wherein the method comprises the administration to said patient a therapeutically effective amount of a compound according to present invention or a pharmaceutically acceptable salt thereof.

In the context of these methods of the invention, diseases and disorders associated with JAK3, BTK, BLK, ITK or TEC are as defined above.

In the context of these methods of the invention, the compounds of the present invention preferably bind covalently to JAK3, BTK, BLK, ITK or TEC, preferably to a cysteine residue.

As used herein, the term “treating” or “treatment” is intended to refer to all processes, wherein there may be a slowing, interrupting, arresting, or stopping of the progression of a disease, but does not necessarily indicate a total elimination of all symptoms.

All embodiments discussed above with respect to the pharmaceutical composition of the invention also apply to the above mentioned first or second medical uses or methods of the invention.

The preparation of compounds of the present invention is described in the literature, like in WO 2011/048082 A1. An exemplary route for the preparation of compounds of the present invention is described below. It is clear to a practitioner in the art to combine or adjust such routes especially in combination with the introduction of activating or protective chemical groups.

An exemplary general route for the preparation of compounds according to the present invention is outlined in Scheme 1. Compound (IV) is used for demonstration purposes. A person skilled in the art would recognise that compound (IV) could be substituted with similar reagents to synthesise further compounds described herein.

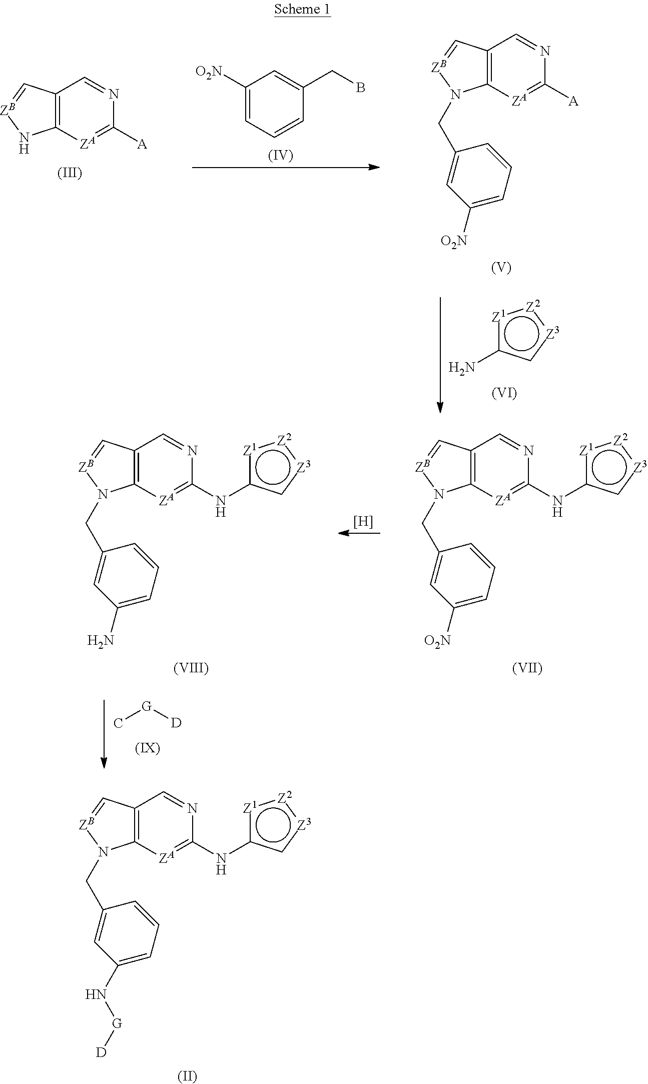

Compounds of formula (II) can be formed from compounds (III), (IV), (VI) and (IX) which are either commercially available or can be made by those skilled in the art. A wide range of solvents are optionally employed for these reactions, including protic solvents such as alcohols; polar aprotic solvents such as dimethylsulfoxide, DMF, acetonitrile, dioxane, THF; non-polar solvents such as toluene, DCM; or basic solvents such as pyridine. The reactions can optionally be promoted by the addition of a base which include but are not limited to amine bases such as triethylamine and DIPEA; or metal carbonates. The reactions can be optionally promoted by acids including mineral acids such as hydrogen chloride; organic acids and Lewis acids. A, B and C are suitable leaving groups such as halogens. G is SO₂ or C(O). D is an optionally substituted alkene or alkyne.

The person skilled in the art would understand that the order of events would depend on the conditions of the reaction and the nature of the reagents; that more than one route to each compound might be possible; that an alternate order of events to those specified in Scheme 1 might be possible.

In one embodiment, a compound of formula (III) is reacted with a compound of formula (IV) in the presence of a base, such as potassium carbonate; in a polar aprotic solvent such as acetonitrile to afford a compound of formula (V). This is then reacted with a compound of formula (VI) in the presence of an acid, such as hydrogen chloride; in a protic solvent such as isopropanol; at a temperature above 20° C. such as 90° C. to give a compound of formula (VII). This is then reacted with a reducing reagent such as hydrogen; in the presence of a catalyst such as PdC; in a protic solvent such as methanol to give a compound of formula (VIII). This is then reacted with a compound of formula (IX) in the presence of a base such as DIPEA; in a non-polar solvent such as DCM to afford a compound of formula (II).

### BRIEF DESCRIPTION OF THE FIGURE

FIG. 1: Amino acid sequence of human JAK3 (1P11P100002773.4). The peptide LVMEYLPSGCLR (position 900-911) with cysteine residue 909 is underlined.

### EXAMPLES

### Analytical Methods

NMR spectra were obtained on a Brucker dpx400. LCMS was carried out on an Agilent 1100 using a Gemini C18, 3×30 mm, 3 micron. Column flow was 1.2 mL/min and solvents used were water and acetonitrile (0.1% formic acid-high pH, 0.1% ammonia-low pH) with an injection volume of 3 μL. Wavelengths were 254 and 210 nm.

### Method A

Column: Phenomenex Gemini-C18, 3×30 mm, 3 microns. Flow rate: 1.2 mL/min

TABLE 1

| Time (min) | Water (%) | ACN (%) |
|---|---|---|
| 0 | 95 | 5 |
| 3 | 5 | 95 |
| 4.5 | 5 | 95 |
| 4.6 | 95 | 5 |

| | |
|---|---|
| 5 | STOP |

### Method B

Column: Phenomenex Gemini-C18, 4.6×150 mm, 5 microns. Flow rate: 1.0 mL/min

TABLE 2

| Time (min) | Water (%) | ACN (%) |
|---|---|---|
| 0.00 | 95.0 | 5.0 |
| 11.00 | 5.0 | 95.0 |
| 13.00 | 5.0 | 95.0 |
| 13.01 | 95.0 | 5.0 |

| | |
|---|---|
| 14.00 | STOP |

### Method C

Column: Phenomenex Gemini-NX C18, 4.6×150 mm, 5 microns. Flow: 1 mL/min. Gradient:

TABLE 3

| Time (min) | Water | Acetonitrile |
|---|---|---|
| 0 | 95 | 5 |
| 11 | 5 | 95 |
| 13 | 5 | 95 |
| 13.01 | 95 | 5 |
| 16.00 | 95 | 5 |

| | |
|---|---|
| 16.01 | STOP |

TABLE 4
Abbreviations

| | |
|---|---|
| ACN | Acetonitrile |
| Ar | Aryl |
| aq | Aqueous |
| Boc | Tert-Butoxycarbonyl |
| BuLi | Butyllithium |
| DCM | Dichloromethane |
| DEAD | Diethyl azodicarboxylate |
| DIAD | Diisopropyl azodicarboxylate |
| DIPEA | Diisopropylethylamine |
| DME | 1,2-Dimethoxyethane |
| DMF | N,N′-Dimethylformamide |
| DMF-DMA | N,N-dimethylformamide dimethylacetal |
| DMSO | N,N′-dimethylsulfoxide |
| DP | Drug pulldown |
| DTT | Dithiothreitol |
| EDC | 1-Ethyl-3-(3-dimethylaminopropyl)carbodiimide |
| EDTA | Ethylenediaminetetraacetic acid |
| EtOAc | Ethyl acetate |
| EtOH | Ethanol |
| eq | Equivalents |
| g | Grams |
| h | Hours |
| HATU | O-(7-Azabenzotriazol-1-yl)-N,N,N′,N′-tetramethyluronium |
| | hexafluorophosphate |
| HCl | Hydrochloric acid |
| HOBt | 1-Hydroxybenzotriazole |
| IC₅₀ | 50% inhibition concentration |
| IPA | Isopropanol |
| iPr | Isopropyl |
| L | Litres |
| LC-MS | Liquid chromatography mass spectroscopy |
| M | Molar |
| MeOH | Methanol |
| Mesyl | Methanesulfonyl chloride |
| mg | Milligrams |
| min | Minutes |
| mL | Millilitres |
| mm | Millimetres |
| mmol | Millimoles |
| mol % | Molar percent |
| μL | Microlitres |
| nm | Nanometres |
| Pd₂(dba)₃ | Tris(dibenzylideneacetone)dipalladium(0) |
| PBS | Phosphate buffered saline |
| Prep. HPLC | Preparative High performance liquid chromatography |
| PyBroP | Bromotripyrrolidinophosphonium hexafluorophosphate |
| rt | Room temperature |
| RT | Retention time |
| sat. | Saturated |
| THF | Tetrahydrofuran |
| tert | Tertiary |
| Xantphos | 4,5-Bis(diphenylphosphino)-9,9-dimethylxanthene |

### EXPERIMENTAL

### Example 1

### N-(3-((6-((1-Methyl-1H-pyrazol-4-yl)amino)-1H-pyrazolo[3,4-d]pyrimidin-1-yl)methyl)phenyl)acrylamide

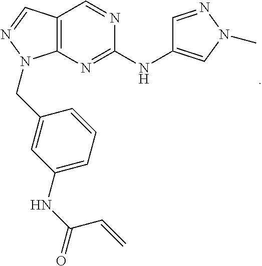 [SMILES: C=CC(=O)Nc1cccc(Cn2ncc3cnc(Nc4cnn(C)c4)nc32)c1]

A suspension of 6-chloro-1H-pyrazolo[3,4-d]pyrimidine (10 g, 52 mmol), potassium carbonate (2 eq) and 1-(bromomethyl)-3-nitrobenzene (1.2 eq) in acetonitrile (150 mL) was stirred at rt for 20 h. The solvent was reduced in vacuo, and the residue dissolved in ethyl acetate and washed with water. The organics were dried (Na₂SO₄), reduced in vacuo and purified by column chromatography to give 6-chloro-1-(3-nitrobenzyl)-1H-pyrazolo[3,4-d]pyrimidine.

To a solution of 6-chloro-1-(3-nitrobenzyl)-1H-pyrazolo[3,4-d]pyrimidine (2.2 g, 7.6 mmol) in isopropanol (62 mL) was added 1-methyl-1H-pyrazol-4-amine (2.5 eq) and concentrated HCl solution (0.2 mL) and the reaction was heated at 90° C. for 18 h. After cooling to rt, the solid was filtered, washed with cold isopropanol and then air dried to give N-(1-methyl-1H-pyrazol-4-yl)-1-(3-nitrobenzyl)-1H-pyrazolo[3,4-d]pyrimidin-6-amine.

To a solution of N-(1-methyl-1H-pyrazol-4-yl)-1-(3-nitrobenzyl)-1H-pyrazolo[3,4-d]pyrimidin-6-amine (0.6 g, 1.7 mmol) in MeOH (25 mL) was added concentrated HCl solution (0.6 mL) and then palladium on carbon (60 mg) and the reaction stirred under a balloon of hydrogen for 2 h. The resulting mixture was filtered through Celite and the filtrate concentrated in vacuo to give 1-(3-aminobenzyl)-N-(1-methyl-1H-pyrazol-4-yl)-1H-pyrazolo[3,4-d]pyrimidin-6-amine.

To a solution of 1-(3-aminobenzyl)-N-(1-methyl-1H-pyrazol-4-yl)-1H-pyrazolo[3,4-d]pyrimidin-6-amine (50 mg, 0.16 mmol) in DCM (imp and DMF (1 mL) was added DIPEA (2 eq) and then acryloyl chloride (1.2 eq) and the reaction was stirred at rt for 16 h. The mixture was diluted with DCM and washed with 1M aq. HCl solution. The organics were washed with sat. aq. NaHCO₃ solution and then water, dried (Na₂SO₄), reduced in vacuo and purified by prep. HPLC to give the title compound. LC-MS method C, (ES+) 375, RT=7.08 min.

### Example 2

### N-(2-Fluoro-5-((6-((1-methyl-1H-pyrazol-4-yl)amino)-1H-pyrazolo[3,4-c]pyrimidin-1-yl)methyl)phenyl)acrylamide

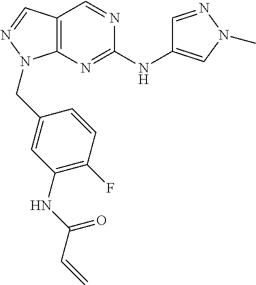 [SMILES: C=CC(=O)Nc1cc(Cn2ncc3cnc(Nc4cnn(C)c4)nc32)ccc1F]

The title compound was made according to the procedure in Example 1 using 4-(bromomethyl)-1-fluoro-2-nitrobenzene. LC-MS method C, (ES+) 393, RT=7.19 min.

### Example 3

### N-(3-((6-((1-Methyl-1H-pyrazol-4-yl)amino)-1H-pyrazolo[3,4-d]pyrimidin-1-yl)methyl)phenyl)propiolamide

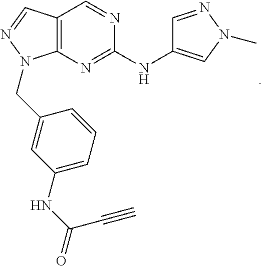 [SMILES: C#CC(=O)Nc1cccc(Cn2ncc3cnc(Nc4cnn(C)c4)nc32)c1]

To a solution of 1-(3-aminobenzyl)-N-(1-methyl-1H-pyrazol-4-yl)-1H-pyrazolo[3,4-d]pyrimidin-6-amine (prepared in Example 1) (50 mg, 0.16 mmol) in DMF (1.5 mL) was added propiolic acid (0.9 eq), DIPEA (3 eq) and then PyBroP (1.3 eq) and the reaction stirred at rt for 3 h. The reaction was diluted with DCM and washed with 1M aq. HCl solution. The organics were washed with sat. aq. NaHCO₃ solution and then water, dried (Na₂SO₄), reduced in vacuo and purified by prep. HPLC to give the title compound. LC-MS method C, (ES+) 373, RT=6.99 min.

### Example 4

### N-(3-((6-((1-Methyl-1H-pyrazol-4-yl)amino)-1H-pyrazolo[3,4-d]pyrimidin-1-yl)methyl)phenyl)ethenesulfonamide

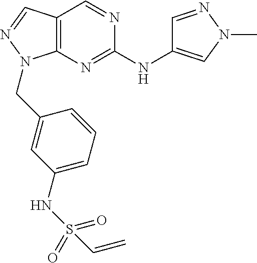 [SMILES: C=CS(=O)(=O)Nc1cccc(Cn2ncc3cnc(Nc4cnn(C)c4)nc32)c1]

To a solution of 2-chloroethanesulfonyl chloride (1.1 eq) in DCM (2 mL) was added diisopropylethylamine (2.5 eq) and the mixture was stored for 30 min. 1-(3-Aminobenzyl)-N-(1-methyl-1H-pyrazol-4-yl)-1H-pyrazolo[3,4-d]pyrimidin-6-amine (prepared in Example 1) (100 mg, 0.32 mmol) and sat. aq. NaHCO₃ solution (2 mL) were added and the reaction stirred at rt for 3 h. The phases were separated and the product extracted into DCM, dried (Na₂SO₄), reduced in vacuo and purified by prep. HPLC to give the title compound. LC-MS method C, (ES+) 411, RT=7.17 min.

### Example 5

### N-(3-((6-((1-Methyl-1H-pyrazol-4-yl)amino)-1H-pyrazolo[3,4-d]pyrimidin-1-yl)methyl)phenyl)methacrylamide

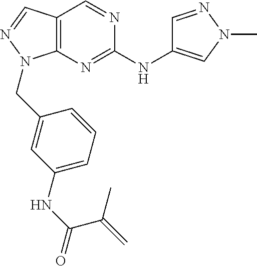 [SMILES: C=C(C)C(=O)Nc1cccc(Cn2ncc3cnc(Nc4cnn(C)c4)nc32)c1]

The title compound was made according to the procedure in Example 1 using methacryloyl chloride. LC-MS method C, (ES+) 389, RT=7.44 min.

### Example 6

### 3-Methyl-N-(3-((6-((1-methyl-1H-pyrazol-4-yl)amino)-1H-pyrazolo[3,4-d]pyrimidin-1-yl)methyl)phenyl)but-2-enamide

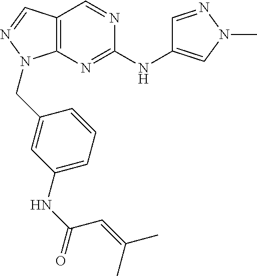 [SMILES: CC(C)=CC(=O)Nc1cccc(Cn2ncc3cnc(Nc4cnn(C)c4)nc32)c1]

The title compound was made according to the procedure in Example 1 using 3-methylbut-2-enoyl chloride.

LC-MS method C, (ES+) 403, RT=7.72 min.

### Example 7

### N-(3-((2-((1-Methyl-1H-pyrazol-4-yl)amino)-7H-pyrrolo[2,3-d]pyrimidin-7-yl)methyl)phenyl)ethenesulfonamide

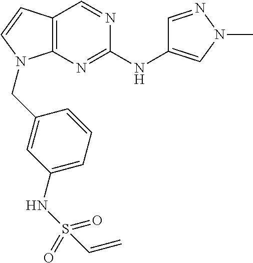 [SMILES: C=CS(=O)(=O)Nc1cccc(Cn2ccc3cnc(Nc4cnn(C)c4)nc32)c1]

A suspension of 2-chloro-7H-pyrrolo[2,3-d]pyrimidine (1 g, 6.5 mmol), potassium carbonate (2 eq) and 1-(bromomethyl)-3-nitrobenzene (1.2 eq) in acetonitrile (150 mL) was stirred at rt for 20 h. The solvent was then reduced in vacuo and the residue diluted with ethyl acetate and washed with water. The organics were dried (Na₂SO₄) and reduced in vacuo to give 2-chloro-7-(3-nitrobenzyl)-7H-pyrrolo[2,3-d]pyrimidine.

To a solution of 2-chloro-7-(3-nitrobenzyl)-7H-pyrrolo[2,3-d]pyrimidine (100 mg, 0.4 mmol) in isopropanol (2 mL) was added 1-methyl-1H-pyrazol-4-amine (2.5 eq) and concentrated HCl solution (0.05 mL) and the mixture was reacted in the microwave at 140° C. for 1 h. The mixture was then filtered, washed with cold isopropanol and then air dried to give N-(1-methyl-1H-pyrazol-4-yl)-7-(3-nitrobenzyl)-7H-pyrrolo[2,3-d]pyrimidin-2-amine.

To a solution of N-(1-methyl-1H-pyrazol-4-yl)-7-(3-nitrobenzyl)-7H-pyrrolo[2,3-d]pyrimidin-2-amine (80 mg, 0.2 mmol) in MeOH (5 mL) was added SnCl₂ and the reaction was heated at 60° C. for 4 h. After cooling to rt the mixture was extracted into ethyl acetate, washed with water and brine, dried (MgSO₄) and reduced in vacuo to give 7-(3-aminobenzyl)-N-(1-methyl-1H-pyrazol-4-yl)-7H-pyrrolo[2,3-d]pyrimidin-2-amine.

To a solution of 7-(3-amino benzyl)-N-(1-methyl-1H-pyrazol-4-yl)-7H-pyrrolo[2,3-d]pyrimidin-2-amine (50 mg, 0.16 mmol) in DCM (1 mL) and DMF (1 mL) was added DIPEA (2 eq) and then acryloyl chloride (1.2 eq) and the reaction stirred at rt for 16 h. The mixture was then diluted with DCM and washed with 1M aq. HCl solution. The organics were then washed with sat. aq. NaHCO₃ solution and water, dried (Na₂SO₄), reduced in vacuo and purified by column chromatography to give the title compound. LC-MS method C, (ES+) 410, RT=5.76 min.

### Example 8

### N-(3-((6-((1-Methyl-1H-pyrazol-3-yl)amino)-1H-pyrazolo[4,3-c]pyridin-1-yl)methyl)phenyl)acrylamide

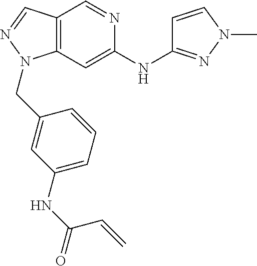 [SMILES: C=CC(=O)Nc1cccc(Cn2ncc3cnc(Nc4ccn(C)n4)cc32)c1]

A suspension of 6-chloro-1H-pyrazolo[4,3-c]pyridine (1 g, 6.5 mmol), potassium carbonate (2 eq) and 1-(bromomethyl)-3-nitrobenzene (1.2 eq) in acetonitrile (30 mL) was stirred at rt for 20 h. The solvent was reduced in vacuo, and the residue dissolved in ethyl acetate and washed with water. The organics were dried (Na₂SO₄), reduced in vacuo and purified by column chromatography to give 6-chloro-1-(3-nitrobenzyl)-1H-pyrazolo[4,3-c]pyridine.

To a degassed solution of Pd₂(dba)₃ (0.1 eq) and XANTPHOS (0.2 eq) in dioxane (5 mL0 was added cesium carbonate (2 eq), 1-methyl-1H-pyrazol-3-amine (1.2 eq) and 6-chloro-1-(3-nitrobenzyl)-1H-pyrazolo[4,3-c]pyridine (100 mg, 0.34 mmol) and the reaction was heated at 110° C. for 18 h. After cooling to rt, the reaction was diluted with ethyl acetate and washed with water. The organics were dried (Na₂SO₄), reduced in vacuo and purified by column chromatography to give N-(1-methyl-1H-pyrazol-3-yl)-1-(3-nitrobenzyl)-1H-pyrazolo[4,3-c]pyridin-6-amine.

To a solution of N-(1-methyl-1H-pyrazol-3-yl)-1-(3-nitrobenzyl)-1H-pyrazolo[4,3-c]pyridin-6-amine (50 mg, 0.14 mmol) in MeOH (5 mL) was added concentrated HCl solution (0.1 mL) and then palladium on carbon (10 mg) and the reaction stirred under a balloon of hydrogen for 2 h. The resulting mixture was filtered through Celite and the filtrate concentrated in vacuo to give 1-(3-aminobenzyl)-N-(1-methyl-1H-pyrazol-3-yl)-1H-pyrazolo[4,3-c]pyridin-6-amine.

To a solution of 1-(3-aminobenzyl)-N-(1-methyl-1H-pyrazol-3-yl)-1H-pyrazolo[4,3-c]pyridin-6-amine (40 mg, 0.13 mmol) in DCM (1 mL) and DMF (1 mL) was added DIPEA (2 eq) and then acryloyl chloride (1.2 eq) and the reaction was stirred at rt for 16 h. The mixture was diluted with DCM and washed with 1M aq. HCl solution. The organics were washed with sat. aq. NaHCO₃ solution and then water, dried (Na₂SO₄), reduced in vacuo and purified by prep. HPLC to give the title compound. LC-MS method C, (ES+) 374, RT=5.38 min.

### Example 9

### N-(3-((2-((1-Methyl-1H-pyrazol-4-yl)amino)-7H-pyrrolo[2,3-d]pyrimidin-7-yl)methyl)phenyl)acrylamide

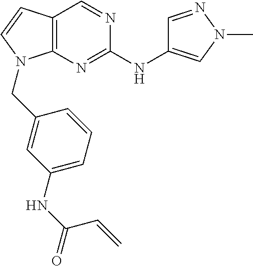 [SMILES: C=CC(=O)Nc1cccc(Cn2ccc3cnc(Nc4cnn(C)c4)nc32)c1]

To a solution of 7-(3-amino benzyl)-N-(1-methyl-1H-pyrazol-4-yl)-7H-pyrrolo[2,3-d]pyrimidin-2-amine (prepared in Example 7) (20 mg, 0.06 mmol) in DCM (1 mL) and DMF (1 mL) was added DIPEA (2 eq) and then acryloyl chloride (1.2 eq) and the reaction was stirred at rt for 16 h. The mixture was diluted with DCM and washed with 1M aq. HCl solution. The organics were washed with sat. aq. NaHCO₃ solution and then water, dried (Na₂SO₄), reduced in vacuo and purified by prep. HPLC to give the title compound. LC-MS method C, (ES+) 374, RT=5.75 min.

### Example 10

### N-(3-((6-((1-Methyl-1H-pyrazol-4-yl)amino)-1H-pyrazolo[4,3-c]pyridin-1-yl)methyl)phenyl)acrylamide

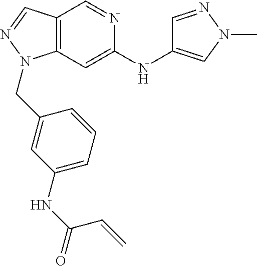 [SMILES: C=CC(=O)Nc1cccc(Cn2ncc3cnc(Nc4cnn(C)c4)cc32)c1]

A suspension of 6-chloro-1H-pyrazolo[4,3-c]pyridine (1 g, 6.5 mmol), potassium carbonate (2 eq) and 1-(bromomethyl)-3-nitrobenzene (1.2 eq) in acetonitrile (30 mL) was stirred at rt for 20 h. The solvent was reduced in vacuo, and the residue dissolved in ethyl acetate and washed with water. The organics were dried (Na₂SO₄), reduced in vacuo and purified by column chromatography to give 6-chloro-1-(3-nitrobenzyl)-1H-pyrazolo[4,3-c]pyridine.

To a degassed solution of Pd₂(dba)₃ (0.1 eq) and XANTPHOS (0.2 eq) in dioxane (5 mL) was added cesium carbonate (2 eq), 1-methyl-1H-pyrazol-4-amine (1.2 eq) and 6-chloro-1-(3-nitrobenzyl)-1H-pyrazolo[4,3-c]pyridine (100 mg, 0.34 mmol) and the reaction was heated at 110° C. for 18 h. After cooling to rt, the reaction was diluted with ethyl acetate and washed with water. The organics were dried (Na₂SO₄), reduced in vacuo and purified by column chromatography to give N-(1-methyl-1H-pyrazol-4-yl)-1-(3-nitrobenzyl)-1H-pyrazolo[4,3-c]pyridin-6-amine.

To a solution of N-(1-methyl-1H-pyrazol-4-yl)-1-(3-nitrobenzyl)-1H-pyrazolo[4,3-c]pyridin-6-amine (50 mg, 0.14 mmol) in MeOH (5 mL) was added concentrated HCl solution (0.1 mL) and then palladium on carbon (10 mg) and the reaction stirred under a balloon of hydrogen for 2 h. The resulting mixture was filtered through Celite and the filtrate concentrated in vacuo to give 1-(3-aminobenzyl)-N-(1-methyl-1H-pyrazol-4-yl)-1H-pyrazolo[4,3-c]pyridin-6-amine.

To a solution of 1-(3-aminobenzyl)-N-(1-methyl-1H-pyrazol-4-yl)-1H-pyrazolo[4,3-c]pyridin-6-amine (25 mg, 0.08 mmol) in DCM (1 mL) and DMF (1 mL) was added DIPEA (2 eq) and then acryloyl chloride (1.2 eq) and the reaction was stirred at rt for 16 h. The mixture was diluted with DCM and washed with 1M aq. HCl solution. The organics were washed with sat. aq. NaHCO₃ solution and then water, dried (Na₂SO₄), reduced in vacuo and purified by prep. HPLC to give the title compound. LC-MS method C, (ES+) 374, RT=5.38 min.

### Reference Example 1

### N-(1-methyl-1H-pyrazol-4-yl)-1-(3-(oxetan-3-ylamino)benzyl)-1H-pyrazolo[3,4-d]pyrimidin-6-amine

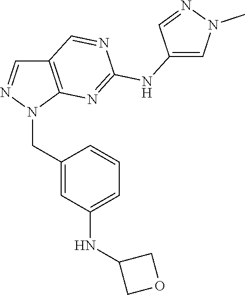 [SMILES: Cn1cc(Nc2ncc3cnn(Cc4cccc(NC5COC5)c4)c3n2)cn1]

This compound was synthesized by procedures analogous to those described above. In addition reference is made to WO 2012/022681 A2, page 121, example 114.

### Reference Example 2

### N-(3-((6-((1-methyl-1H-pyrazol-4-yl)amino)-1H-pyrazolo[3,4-d]pyrimidin-1-yl)methyl)phenyl)acetamide

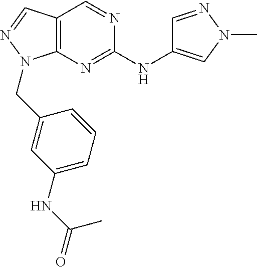 [SMILES: CC(=O)Nc1cccc(Cn2ncc3cnc(Nc4cnn(C)c4)nc32)c1]

This compound was synthesized by procedures analogous to those described above.

### Reference Example 3

### JAK3 inhibitor CP 690,550 (Changelian et al., 2003, Science 302(5646):875-888)

### Biological Assays

Determination of the Effect of the Compounds According to the Invention on Janus Kinases (JAK family) in Kinobeads™ Assays with Immunodetection of Kinases

### Principle of the Assay

The compounds of the present invention as described in the previous examples were tested in a Kinobeads™ assay as described for ZAP-70 (WO-A 2007/137867). Briefly, test compounds (at various concentrations) and the affinity matrix with the immobilized aminopyrido-pyrimidine ligand 24 were added to cell lysate aliquots and allowed to bind to the proteins in the lysate sample. After the incubation time the beads with captured proteins were separated from the lysate. Bound proteins were then eluted and the presence of JAK1; JAK2, JAK3 and TYK2 was detected and quantified using specific antibodies in a dot blot procedure and the Odyssey infrared detection system. Dose response curves for individual kinases were generated and IC₅₀ values calculated. Kinobeads™ assays for ZAP-70 (WO-A 2007/137867) and for kinase selectivity profiling (WO-A 2006/134056) have been previously described.

### Protocols

### Washing of Affinity Matrix

The affinity matrix was washed two times with 15 mL of 1×DP buffer containing 0.2% NP40 (IGEPAL® CA-630, Sigma, #13021) and then resuspended in 1×DP buffer containing 0.2% NP40 (3% beads slurry).

5×DP buffer: 250 mM Tris-HCl pH 7.4, 25% Glycerol, 7.5 mM MgCl₂, 750 mM NaCl, 5 mM Na₃VO₄; filter the 5×DP buffer through a 0.22 μm filter and store in aliquot at −80° C. The 5×DP buffer is diluted with H₂O to 1×DP buffer containing 1 mM DTT and 25 mM NaF.

### Preparation of Test Compounds

Stock solutions of test compounds were prepared in DMSO. In a 96 well plate 30 μL solution of diluted test compounds at 5 mM in DMSO were prepared. Starting with this solution a 1:3 dilution series (9 steps) was prepared. For control experiments (no test compound) a buffer containing 2% DMSO was used.

Cell culture and Preparation of Cell Lysates

Molt4 cells (ATCC catalogue number CRL-1582) and Ramos cells (ATCC catalogue number CRL-1596) were grown in 1 L Spinner flasks (Integra Biosciences, #182101) in suspension in RPMI 1640 medium (Invitrogen, #21875-034) supplemented with 10% Fetal Bovine Serum (Invitrogen) at a density between 0.15×10⁶ and 1.2×10⁶ cells/mL. Cells were harvested by centrifugation, washed once with 1×PBS buffer (Invitrogen, #14190-094) and cell pellets were frozen in liquid nitrogen and subsequently stored at −80° C. Cells were homogenized in a Potter S homogenizer in lysis buffer: 50 mM Tris-HCl, 0.8% NP40, 5% glycerol, 150 mM NaCl, 1.5 mM MgCl₂, 25 mM NaF, 1 mM sodium vanadate, 1 mM DTT, pH 7.5. One complete EDTA-free tablet (protease inhibitor cocktail, Roche Diagnostics, 1873580) per 25 mL buffer was added. The material was dounced 10 times using a mechanized POTTER S, transferred to 50 mL falcon tubes, incubated for 30 minutes on ice and spun down for 10 minutes at 20,000 g at 4° C. (10,000 rpm in Sorvall SLA600, precooled). The supernatant was transferred to an ultracentrifuge (UZ)-polycarbonate tube (Beckmann, 355654) and spun for 1 hour at 100.000 g at 4° C. (33.500 rpm in Ti50.2, precooled). The supernatant was transferred again to a fresh 50 mL falcon tube, the protein concentration was determined by a Bradford assay (BioRad) and samples containing 50 mg of protein per aliquot were prepared. The samples were immediately used for experiments or frozen in liquid nitrogen and stored frozen at −80° C.

### Dilution of Cell Lysate

Cell lysate (approximately 50 mg protein per plate) was thawed in a water bath at room temperature and then stored on ice. To the thawed cell lysate 1×DP 0.8% NP40 buffer containing protease inhibitors (1 tablet for 25 mL buffer; EDTA-free protease inhibitor cocktail; Roche. Diagnostics 1873580) was added in order to reach a final protein concentration of 10 mg/mL total protein. The diluted cell lysate was stored on ice. Mixed Molt4Ramos lysate was prepared by combining one volume of Molt4 lysate and two volumes of Ramos lysate (ratio 1:2).

Incubation of Lysate with Test Compound and Affinity Matrix

To a 96 well filter plate (Multiscreen HTS, BV Filter Plates, Millipore #MSBVN1250) were added per well: 100 μL affinity matrix (3% beads slurry), 3 μL of compound solution, and 50 μL of diluted lysate. Plates were sealed and incubated for 3 hours in a cold room on a plate shaker (Heidolph tiramax 1000) at 750 rpm. Afterwards the plate was washed 3 times with 230 μL washing buffer (1×DP 0.4% NP40). The filter plate was placed on top of a collection plate (Greiner bio-one, PP-microplate 96 well V-shape, 65120) and the beads were then eluted with 20 μL of sample buffer (100 mM Tris, pH 7.4, 4% SDS, 0.00025% bromophenol blue, 20% glycerol, 50 mM DTT). The eluate was frozen quickly at −80° C. and stored at −20° C.

### Detection and Quantification of Eluted Kinases

The kinases in the eluates were detected and quantified by spotting on nitrocellulose membranes and using a first antibody directed against the kinase of interest and a fluorescently labelled secondary antibody (anti-rabbit IRDye™ antibody 800 (Licor, #926-32211). The Odyssey Infrared Imaging system from LI-COR Biosciences (Lincoln, Nebr., USA) was operated according to instructions provided by the manufacturer (Schutz-Geschwendener et al., 2004. Quantitative, two-color Western blot detection with infrared fluorescence. Published May 2004 by LI-COR Biosciences, www.licor.com). After spotting of the eluates the nitrocellulose membrane (BioTrace NT; PALL, #BTNT30R) was first blocked by incubation with Odyssey blocking buffer (LICOR, 927-40000) for one hour at room temperature. Blocked membranes were then incubated for 16 hours at the temperature shown in table 5 with the first antibody diluted in Odyssey blocking buffer (LICOR #927-40000). Afterwards the membrane was washed twice for 10 minutes with PBS buffer containing 0.2% Tween 20 at room temperature. The membrane was then incubated for 60 minutes at room temperature with the detection antibody (anti-rabbit IRDye™ antibody 800, Licor, #926-32211) diluted in Odyssey blocking buffer (LICOR #927-40000). Afterwards the membrane was washed twice for 10 minutes each with 1×PBS buffer containing 0.2% Tween 20 at room temperature. Then the membrane was rinsed once with PBS buffer to remove residual Tween 20. The membrane was kept in PBS buffer at 4° C. and then scanned with the Odyssey instrument. Fluorescence signals were recorded and analysed according to the instructions of the manufacturer.

TABLE 5
Sources and dilutions of antibodies

| | | Temperature of | |
|---|---|---|---|
| Target | Primary antibody | primary | Secondary |
| kinase | (dilution) | incubation | antibody (dilution) |
| JAK1 | Call signalling #3332 | 4° C. | Licor anti-rabbit 800 |
| | (1:100) | | (1:15000) |
| JAK2 | Cell signalling #3230 | Room | Licor anti-rabbit 800 |
| | (1:100) | temperature | (1:15000) |
| JAK3 | Cell signalling #3775 | 4° C. | Licor anti-rabbit 800 |
| | (1:100) | | (1:5000) |
| TYK2 | Cell signalling #06-638 | Room | Licor anti-rabbit 800 |
| | (1:1000) | temperature | (1:5000) |

### Results

Table 6 provides data for selected compounds of the invention in the JAK Kinobeads™ assay.

TABLE 6
Inhibition values (IC₅₀ in nM) as determined in the Kinobeads ™ assay
with antibody detection.

| Example | JAK1 | JAK2 | JAK3 | Tyk2 |
|---|---|---|---|---|
| 1 | >10000 | >10000 | 5 | >10000 |
| 2 | >10000 | >10000 | 16 | >10000 |
| 3 | >10000 | >10000 | 10 | 6958 |
| 4 | >10000 | >10000 | 10 | 6132 |
| 5 | >10000 | >10000 | 57 | >10000 |
| 6 | >10000 | >10000 | 1309 | >10000 |
| 7 | 7694 | >10000 | 12 | 901 |
| 8 | >10000 | >10000 | 5 | >10000 |
| 9 | 9420 | >10000 | 3 | 8083 |
| 10 | 5368 | >10000 | 1 | 5080 |
| Reference | >10000 | >10000 | 206 | >10000 |
| example 2 | | | | |

Kinase Selectivity Profiling of Compounds in Kinobeads™ Assays with Quantitative Mass Spectrometry Detection of Kinases

Selected compounds of the present invention as described in the previous examples were tested in a Kinobeads™ assay as described (WO-A 2006/134056; Bantscheff et al., 2007. Nature Bioteclmol. 25, 1035-1044).

Compounds were incubated with cell lysate aliquots (1:1 mix of Jurkat and Ramos cell lysates) for 45 minutes at 4° C. and allowed to bind to the proteins in the lysate sample. Then the Kinobeads™ affinity matrix was added to capture proteins that were not interacting with the previously added compound. After this two hour incubation step at 4° C. beads were separated from the lysate and bead bound proteins were eluted in SDS sample buffer and subsequently separated by SDS-Polyacrylamide gel electrophoresis. The gel was stained with colloidal Coomassie and stained areas of each gel lane were cut out and subjected to in-gel proteolytic digestion with trypsin. Peptides originating from the different gel areas were labeled with isobaric tagging reagents (TMT reagents, Thermofisher) as shown in Table 7. The TMT reagents are a set of multiplexed, amine-specific, stable isotope reagents that can label peptides in up to six different biological samples enabling simultaneous identification and quantitation of peptides. The combined samples were fractionated using reversed-phase chromatography at pH 11 and fractions were subsequently analyzed with a nano-flow liquid chromatography system coupled online to a tandem mass spectrometer (LC-MSMS) experiment followed by reporter ion quantification in the MSMS spectra (Ross et al., 2004. Mol. Cell. Proteomics 3(12):1154-1169; Dayon et al., 2008. Anal. Chem. 80(8):2921-2931; Thompson et al., 2003. Anal. Chem. 75(8):1895-1904). Further experimental protocols can be found in WO2006/134056 and previous publications (Bantscheff et al., 2007. Nat. Biotechnol. 25, 1035-1044; Bantscheff et al., 2011. Nat. Biotechnol. 29(3):255-265).

TABLE 7
Labeling of peptides with TMT isobaric tagging reagents

| Sample | Compound concentration (nM) | TMT6 reagent |
|---|---|---|
| 1 | 3000 | 126 |
| 2 | 750 | 127 |
| 3 | 187 | 128 |
| 3 | 47 | 129 |
| 5 | 12 | 130 |
| 6 | 0 | 131 |

As shown in Table 8, the kinase selectivity profile of selected compounds of the invention was determined in kinobeads assays with mass spectrometry detection of kinases as described previously (Bantscheff et al., 2007. Nat. Biotechnol. 25(9):1035-1044; WO-A 2006/134056).

TABLE 8
Inhibition values (IC₅₀ in nM) as determined in the Kinobeads ™ assay
with quantitative mass spectrometry.

| Example | BTK | TEC | BLK | ITK |
|---|---|---|---|---|
| 1 | 12 | 12 | 20 | 86 |
| 2 | 72 | 34 | 406 | 818 |
| 3 | 12 | n.d. | 12 | 12 |

(n.d. = not determined)

### Determination of the Effect of the Compounds According to the Invention on BLK, BTK, ITK and JAK3 in Kinase Assays

A radiometric protein kinase assay (³³PanQinase® Activity Assay; ProQinase GmbH, Freiburg, Germany) was used for measuring the kinase activity of the protein kinases BLK, BTK, ITK and JAK3. All kinase assays were performed in 96-well FlashPlates™ from PerkinElmer (Boston, Mass., USA) in a 50 μl reaction volume. The reaction cocktail was pipetted in four steps in the following order:
20 μl of assay buffer (standard buffer)
5 μl of ATP solution (in H₂O)
5 μl of test compound (in 10% DMSO)
10 μl of substrate/10 μl of enzyme solution (premixed)

The assay for all enzymes contained 70 mM HEPES-NaOH, pH 7.5, 3 mM MgCl₂, 3 mM MnCl₂, 3 μM Na-orthovanadate, 1.2 mM DTT, ATP/[γ-³³P]-ATP (variable amounts, corresponding to the apparent ATP-Kₘ of the respective kinase), protein kinase, and substrate (Table 9).

The reaction cocktails were incubated at 30° C. for 60 minutes. The reaction was stopped with 50 μl of 2% (v/v) H₃PO₄, plates were aspirated and washed two times with 200 μl 0.9% (w/v) NaCl. Incorporation of ³³Pi was determined with a microplate scintillation counter (Microbeta, Wallac). All assays were performed with a BeckmanCoulter/SAGIAN™ Core System.

TABLE 9
Enzymes and substrates

| | Kinase | ATP | | Substrate |
|---|---|---|---|---|
| | concen- | concen- | | concen- |
| | tration | tration | | tration |
| Kinase | ng/50 μl | μM | Substrate | μg/50 μl |
| BTK | 20 | 3.0 | poly(Glu, Tyr)4:1 | 0.125 |
| ITK | 50 | 1.0 | poly(Glu, Tyr)4:1 | 0.125 |
| JAK3 | 100 | 0.3 | poly(Ala, GLU, Lys, Tyr)6:2:5:1 | 0.125 |

Table 10 provides data for selected compounds of the invention in kinase assays.

TABLE 10
Proquinase enzyme assay data (IC₅₀ values in nM)

| Example | BLK | BTK | ITK | JAK3 |
|---|---|---|---|---|
| 1 | nd | 20 | 230 | 3 |
| 2 | 2000 | 323 | n.d. | n.d. |
| 3 | n.d. | 1 | 3 | 4 |
| 4 | n.d. | 20 | 80 | 7 |
| Reference | n.d. | 2000 | inactive | 290 |
| example 2 | | | | |

(n.d. = not determined)

### Cell Assays

pSTAT5 Assay

### Assay Principle

STAT5 phosphorylation represents one of the proximal events in the signalling cascade downstream of JAK3 activation. Therefore STAT5 phosphorylation is an appropriate readout to assess the mechanistic effect of JAK3 inhibition. Stimulation of human YT cells, an NK-like cell line, with interleukin-2 (IL-2) results in phosphorylation of STATS at tyrosine residue 694 (Tyr694) that can be quantitatively measured by immunodetection with specific antibodies and an appropriate detection method, in this case AlphaScreen assay technology.

### Assay Protocol

### Cell Culture and Cell Seeding

Human YT cells were grown in RPMI medium (Lonza, BE12-167) with 2 mM L-Glutamine (Invitrogen, 25030-024) and 10% heat-inactivated FBS (Invitrogen, 10106-169) and kept in a humidified incubator (37° C., 5% CO₂). Cells were harvested by centrifugation, washed once with HBSS (Invitrogen, 14180-046), resuspended in HESS at 1.5×10⁶ cells/ml and 0.9×10⁴ cells were seeded in 6 μl per well in a 96 well White plate (PerkinElmer, 6005569).

Treatment with Test Compounds and 1 L-2 Stimulation

Test compounds were dissolved in DMSO and a 1:3 dilution series (9 steps) was prepared. To generate a dose response curve, 41 of fourfold concentrated compound in 4% DMSO/HBSS were added to each cell sample in the 96 well plate resulting in a final DMSO concentration of 1% DMSO. Cells were incubated for one hour in a humidified incubator (37° C., 5% CO₂). To each well 3 μl of a fourfold concentrated IL-2 solution (Recombinant human IL-2, Peprotech 200-02; 120 nM solution in HBSS) was added and incubated for 30 minutes at room temperature. Cells were lysed by adding 3 μl of 5× lysis buffer (SureFire lysis buffer; Perkin Elmer, TGRS5S10K) and incubated for 10 minutes at room temperature with gentle shaking.

### Signal Detection

For signal detection by AlphaScreen® technology the SureFire phospho-STAT5 (Tyr694Tyr699) kit was used according to instructions provided by the manufacturer (Perkin Elmer, TGRS5S10K). Acceptor beads were added as recommended by the manufacturer (Reactivation buffer/Activation buffer/Acceptor beads at a ratio of 40:10:1) and incubated at room temperature for 1.5 hours with gentle shaking. Then donor beads were added as recommended (Dilution buffer/Donor beads at a ratio of 20:1) and incubated at room temperature for 1.5 hours with gentle shaking. Plates were read on an Envision instrument (Perkin Elmer) with the AlphaScreen protocol. Data were analysed in BioAssay using the nonlinear regression for a sigmoidal dose-response with a variable slope.

Table 11 provides data for selected compounds of the invention in the pSTAT5 cell assay.

TABLE 11
pSTAT5 cell assay data (IC₅₀ values in nM)

| Example | pSTAT5 |
|---|---|
| 1 | 31 |
| 2 | 378 |
| 3 | 33 |
| 4 | 74 |
| 5 | 2005 |
| 7 | 79 |

Cell Assay with Washout of Compounds

### Assay Principle

This time course experiment allows determination as to whether the pharmacological effect of the tested compounds persists over time even after the compound is removed from the cell samples.

### Assay Protocol

### Cell Culture and Cell Seeding

Human YT cells were grown as described above. Cells were harvested by centrifugation and resuspended in RPMI0.5% heat-inactivated FBS. 3×10⁵ cells were seeded in 60 μl per well in a round-bottomed 96 well plate (BD-Falcon, 353077).

Treatment with Test Compounds and 1 L-2 Stimulation

Test compounds were dissolved in DMSO and a 1:3 dilution series (9 steps) was prepared. To generate a dose response curve, 30 μl of fourfold concentrated compound in 4% DMSO/RPMI/0.5% FBS were added to each cell sample in the 96 well plate resulting in a final DMSO concentration of 1% DMSO. Cells were incubated for one hour in a humidified incubator (37° C., 5% CO₂). After incubation cells were washed twice by centrifugation and replacement of media (RPMI/0.5% FBS), with the exception of the time 0 plate. Washed cells were incubated for 30 min, 1, 2 and 4 hours in a humidified incubator (37° C., 5% CO₂) prior to stimulation with IL-2. To each well 30 μl of a fourfold concentrated IL-2 solution (Recombinant human IL-2, Peprotech 200-02; 120 nM solution in RPMI) was added and incubated for 30 minutes at room temperature. Cells were lysed by adding 30 μl of 5× lysis buffer (MSD lysis buffer) and incubated for 10 minutes at 4° C. with gentle shaking.

### Signal Detection

The effect of compounds on STATS phosphorylation was determined in lysates using the MSD MULTI-SPOT® 96 4-spot phosphor-STAT5a,b whole cell lysatc kit (MesoScale Discovery, K150IGD-3) according to the manufacturer's protocol. Data were analysed in BioAssay using the nonlinear regression for a sigmoidal dose-response with a variable slope and IC₅₀ values were determined.

### Results

For reference example 3 (JAK3 inhibitor CP690,550) a significant drop in activity is observed 30 minutes after the washing of cells and inhibition activity is completely lost after 1 hour. The activity of reference example 2 is completely lost 30 minutes after the washing. By contrast, examples 1 and 3 retain activity for up to 4 hours after washing with some drop in activity.

Table 12 provides data for selected compounds of the invention and reference compounds in the cell washout study.

TABLE 12
Time course of pSTAT5 inhibition (pIC50 values) in IL-2 stimulated YT
cells after washout of compounds.

| | 0 min | | | | |
|---|---|---|---|---|---|
| Example | (no wash) | 30 min | 1 hour | 2 hours | 4 hours |
| Reference example 3 | 7.6, 7.8 | 5.1 | inactive | inactive | Inactive |
| Reference example 1 | 5.8, 5.7 | inactive | inactive | inactive | inactive |
| Example 1 | 8.3, 7.6 | 6.7 | 7.2, 6.5 | 7.2 | 7.0 |
| Example 3 | 7.5, 6.4 | 6.2 | 6.9, 6.2 | 7.1 | 6.9 |

### Mass Spectrometry Identification of a JAK3 Peptide Modified by Compound Example 1

### Principle of the Assay

Mass spectrometry analysis of immunoprecipitated JAK3 after compound treatment was used to determine whether compound example 1 covalently binds to JAK3. Jurkat cell lysate was pre-incubated with 10 μM of compound example 1 for 45 minutes. Control samples were incubated without compound (DMSO controls). Subsequently JAK3 was immunoprecipitated with an anti-JAK3 antibody (Abeam ab45141). In addition, a control experiment was performed with anti-IgG (mock immunoprecipitation). The precipitated proteins were separated by SDS-polyacrylamide gel electrophoresis. The gel was stained with colloidal Coomassie and stained areas of each gel lane were cut out and subjected to in-gel proteolytic digestion with trypsin. The peptides originating from the three samples were labeled with iTRAQ reagents as shown in Table 13 and the combined samples were analyzed with a nano-flow liquid chromatography system coupled online to a tandem mass spectrometer (LC-MS/MS) experiment followed by iTRAQ reporter ion quantification in the MSMS spectra (Ross et al., 2004. Mol. Cell. Proteomics 3(12):1154-1169). The gel area containing JAK3 was analysed for 270 minutes with HCDiq (high mass accuracy MSMS spectra) on a Orbitrap Velos mass spectrometer. For the Mascot search standard parameters plus a variable modification of cysteine by compound example 1 was used. Further experimental protocols can be found in WO2006/134056 and a previous publication (Bantschcff et al., 2007. Nature Biotechnology 25, 1035-1044).

TABLE 13
Labeling of peptides with iTRAQ isobaric tagging reagents

| Sample | Compound | Antibody | iTRAQ reagent |
|---|---|---|---|
| 1 | 10 μM example 1 | anti-JAK3, | 115 |
| | | Abcam ab45141 | |
| 2 | 0.25% DMSO | anti-JAK3, | 116 |
| | | Abcam ab45141 | |
| 3 | 0.25% DMSO | rabbit IgG, | 117 |
| | | Sigma I5006 | |

### Protocols

### 1. Preparation of Cell Lysate

Jurkat cells (ATCC catalogue number TIB-152 Jurkat, clone E6-1) were grown in one litre Spinner flasks (Integra Biosciences, #182101) in suspension in RPMI 1640 medium (Invitrogen, #21875-034) supplemented with 10% Fetal Bovine Serum (Invitrogen) at a density between 0.15×10⁶ and 1.2×10⁶ cells/ml. Cells were harvested by centrifugation, washed once with 1×PBS buffer (Invitrogen, #14190-094) and cell pellets were frozen in liquid nitrogen and subsequently stored at −80° C. Jurkat cells were homogenized in a Potter S homogenizer in lysis buffer: 50 mM Tris-HCl, 0.8% NP40, 5% glycerol, 150 mM NaCl, 1.5 mM MgCl₂, 25 mM NaF, 1 mM sodium vanadate, 1 mM DTT, pH 7.5. One complete EDTA-free tablet (protease inhibitor cocktail, Roche Diagnostics, 0.1873580) per 25 ml buffer was added. The material was dounced 10 times using a mechanized POTTER S, transferred to 50 ml falcon tubes, incubated for 30 minutes on ice and spun down for 10 min at 20,000 g at 4° C. (10,000 rpm in Sorvall SLA600, precooled). The supernatant was transferred to an ultracentrifuge (UZ)-polycarbonate tube (Beckmann, 355654) and spun for 1 hour at 100.000 g at 4° C. (33.500 rpm in Ti50.2, precooled). The supernatant was transferred again to a fresh 50 ml falcon tube, the protein concentration was determined by a Bradford assay (BioRad) and samples containing 50 mg of protein per aliquot were prepared. The samples were immediately used for experiments or frozen in liquid nitrogen and stored frozen at −80° C.

### 2. Immunoprecipitation of Proteins

### Coupling of Antibodies

Antibodies were covalently coupled to activated beaded agarose through primary amines. The AminoLink® Plus Coupling Reaction (Thermo Scientific Inc., Rockford, Ill. 61105, USA) involves spontaneous formation of Schiff base bonds between aldehydes (on the support) and amines (on the antibody) and their subsequent stabilization by incubation with a mild reductant (sodium cyanoborohydride). 200 μl AminoLink® resin (Thermo Scientific Inc., 20501) were coupled in one batch at an appropriate concentration with the anti-JAK3 antibody (80 μl Abcam ab45141, Lot GR5571-5). For the mock immunoprecipitation an appropriate amount of rabbit IgG (80 μg Sigma 15006) was coupled to 100 μl AminoLink® resin (Thermo Scientific Inc., 20501).

The AminoLink® resin was washed three times with 10 bead volumes of PBS and subsequently the antibody solution was added to the resin in a 1.5 ml siliconized microfuge tube. 1M NaCNBH₃ (Thermo Scientific Inc., 44892) was prepared freshly in 0.01M NaOH (prepared from 1M NaOH, Merck, 109137) and 25 μl were added per 1 ml reaction volume. The mixture was incubated overnight at 4° C. rotating (NeoLab Rotator, 2-1175). A small amount of the supernatant was kept to determine the coupling efficiency by a Bradford Assay, the rest was discarded. The beads were washed twice with 10 bead volumes of 1 M Tris pH 7.4 (Sigma-Aldrich, S5150). 1 M Tris pH 7.4 was added to the beads at a ratio of 1:1, 25 μl freshly prepared NaCNBH₃ per 1 ml reaction volume was added and incubated rotating for 30 minutes at room temperature. The supernatant was discarded and the beads were washed two times with 10 bead volumes of 1 M NaCl (prepared from 5 M NaCl, Sigma-Aldrich, S5150). Before usage the beads were washed twice with lysis buffer (without DTT) with 0.2% NP40.

Incubation of Cell Lysate with Compound and Immunoprecipitation

The cell lysate was thawn, diluted 1:1 with lysis buffer without DTT and NP40 and further diluted to 5 mg/ml protein concentration with lysis buffer containing 0.4% NP40 (no DTT).

The lysate was transferred to an ultracentrifuge tube (Beckmann, 355654) and centrifuged for 20 minutes at 100.000×g at 4° C. (33.500 rpm in Ti50.2, pre-cooled). The supernatant was transferred into a fresh falcon tube.

Meanwhile a 4 mM solution of the compound was prepared by diluting the 30 mM stock solution with DMSO. Five μl of the 4 mM compound solution and 2 ml ultracentrifuged lysate (10 mg protein per sample) were incubated in 15 ml Greiner tubes on an end-over-end shaker (Roto Shake Genie, Scientific Industries Inc.) for 45 minutes at 4° C. This corresponds to a final concentration of 10 μM of compound example 1. For the control experiments, 0.25% DMSO were used.

After the incubation step the affinity matrix (AminoLink® resin with the immobilized antibody; 100 μl beads per immunoprecipitation sample) was then incubated with the lysate samples on an end-over-end shaker for 1 hour at 4° C. Beads were collected by centrifugation at 2000 rpm for 2 minutes, a small amount of the non-bound fraction was kept and the remaining supernatant was discarded. The beads were transferred to Mobicol-columns (MoBiTech, 10055) with 600 μl lysis buffer (no DTT) and washed with 10 ml lysis buffer containing 0.2% NP40 detergent, followed by 5 ml lysis buffer without detergent. To elute bound proteins, 80 μl 2×SDS sample buffer was added to the column. The column was incubated for 10 minutes at 95° C. and the eluate was transferred to a siliconized microfuge tube by centrifugation. The proteins were then reduced with 50 mM DTT and afterwards alkylated with 108 mM iodoacetamide. Proteins were then separated by SDS-Polyacrylamide electrophoresis (SDS-PAGE).

### 3. Protein Identification by Mass Spectrometry

### 3.1 Protein Digestion Prior to Mass Spectrometric Analysis

Gel-separated proteins were digested in-gel essentially following a previously described procedure (Shevchenko et al., 1996, Anal. Chem. 68:850-858). Briefly, gel-separated proteins were excised from the gel using a clean scalpel, destained twice using 100 μl 5 mM triethylammonium bicarbonate buffer (TEAB; Sigma T7408) and 40% ethanol in water and dehydrated with absolute ethanol. Proteins were subsequently digested in-gel with porcine trypsin (Promega) at a protease concentration of 10 ng/μl in 5 mM TEAB. Digestion was allowed to proceed for 4 hours at 37° C. and the reaction was subsequently, stopped using 5 μl 5% formic acid.

### 3.2 Sample Preparation Prior to Analysis by Mass Spectrometry

Gel plugs were extracted twice with 20 μl 1% formic acid and three times with increasing concentrations of acetonitrile. Extracts were subsequently pooled with acidified digest supernatants and dried in a vacuum centrifuge.

3.3 iTRAQ Labeling of Peptide Extracts

The peptide extracts of samples treated with 200 μM of free example 1 and the solvent control (0.5% DMSO) were treated with different variants of the isobaric tagging reagent (iTRAQ Reagents Multiplex Kit, part number 4352135, Applied Biosystems, Foster City, Calif., USA). The iTRAQ reagents are a set of multiplexed, amine-specific, stable isotope reagents that can label peptides on amino groups in up to four different biological samples enabling simultaneous identification and quantitation of peptides. The iTRAQ reagents were used according to instructions provided by the manufacturer. The samples were resuspended in 10 μl 50 mM TEAB solution, pH 8.5 and 10 μl ethanol were added. The iTRAQ reagent was dissolved in 120 μl ethanol and 10 μl of reagent solution were added to the sample. The labeling reaction was performed at room temperature for one hour on a horizontal shaker and stopped by adding 5 μl of 100 mM TEAB and 100 mM glycine in water. The two labeled sampled were then combined, dried in a vacuum centrifuge and resuspended in 10 μl of 0.1% formic acid in water.

### 3.4 Mass Spectrometric Data Acquisition

Peptide samples were injected into a 1D+, Eksigent) nano LC system which was directly coupled to a Thermo OrbitrapVelos mass spectrometer. Peptides were separated on the LC system using a gradient of aqueous and organic solvents (see below). Solvent A was 0.1% formic acid and solvent B was 70% acetonitrile in 0.1% formic acid.

TABLE 14
Peptide elution off the LC system

| Time (min) | % A | % B |
|---|---|---|
| 0 | 95 | 5 |
| 230 | 40 | 60 |
| 240 | 10 | 90 |
| 250 | 10 | 90 |
| 251 | 95 | 5 |
| 260 | 95 | 5 |

### 3.5 Protein Identification and Quantitation

The peptide mass and fragmentation data generated in the LC-MSMS experiments were used to query a protein data base consisting of an in-house curated version of the International Protein Index (IPI) protein sequence database combined with a decoy version of this database (Elias and Gygi, 2007. Target-decoy search strategy for increased confidence in large-scale protein identifications by mass spectrometry. Nature Methods 4, 207-214). Proteins were identified by correlating the measured peptide mass and fragmentation data with data computed from the entries in the database using the software tool Mascot (Perkins et al., 1999. Probability-based protein identification by searching sequence databases using mass spectrometry data. Electrophoresis 20, 3551-3567). Mascot search parameter were 20 ppm peptide mass tolerance. Fragment mass tolerance 20 mmu. Enzyme trypsin. Fixed modifications iTRAQ (K). Variable modifications iTRAQ (N-term) & Acetyl (N-term) & Oxidation (M) & Carbamidomethyl (C) & compound example 1 (C). Max missed cleavages 3. Protein acceptance thresholds were adjusted to achieve a false discovery rate of below 1% as suggested by hit rates on the decoy data base (Elias and Gygi, 2007. Target-decoy search strategy for increased confidence in large-scale protein identifications by mass spectrometry. Nature Methods 4, 207-214). Relative protein quantitation was performed using peak areas of iTRAQ reporter ion signals essentially as described in an earlier publication (Bantscheff et al., 2007. Nature Biotechnology 25, 1035-1044).

### Results

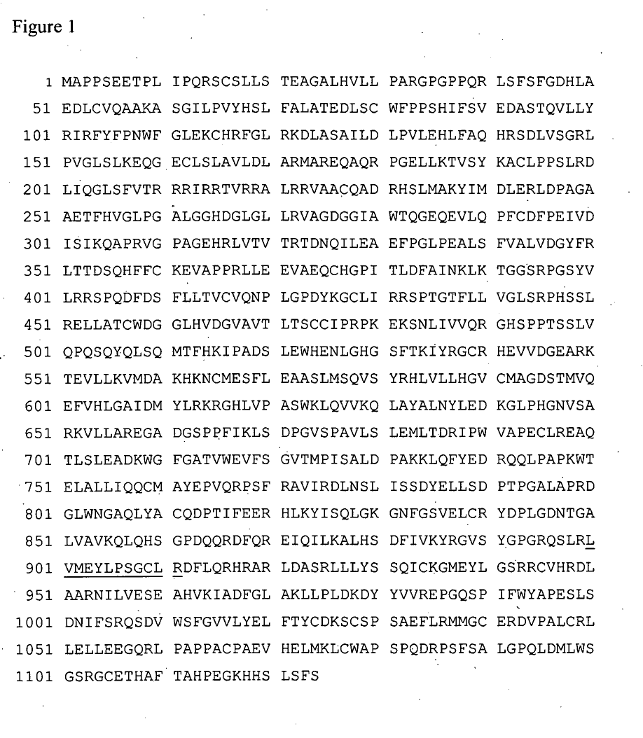

FIG. 1 shows the sequence of human JAK3. The peptide LVMEYLPSGCLR (JAK3 position 900-911) is the only peptide covalently modified by compound example 1. This peptide is modified at cysteine 909 (see Tables 15 to 17).

TABLE 15
Identification of peptide LVMEYLPSGCLR (amino acid residues 900-911 in JAK3
sequence). Fixed modifications: 4TRAQ (K). Variable modifications: 4TRAQ (N-term),
Acetyl (Protein N-term), Carbamidomethyl (C), compound example 1 (C), Oxidation (M).
Cleavage by Trypsin: cuts C-terminal side of K or R unless next residue is P.

| Position | Observed | Expected | Calculated | | |
|---|---|---|---|---|---|
| in JAK3 | mass | mass | mass | ppm | Peptide sequence |
| 900-911 | 799.4131 | 1596.8117 | 1596.8126 | 0 | R.LVMEYLPSGCLR.D |
| | | | | | 4TRAQ (N-term); |
| | | | | | Carbamidomethyl (C); Oxidation |
| | | | | | (M) (Ions score 29) |
| 900-911 | 633.6586 | 1897.9539 | 1897.9565 | 0 | R.LVMEYLPSGCLR.D |
| | | | | | 4TRAQ (N-term); compound |
| | | | | | example 1 (C) (Ions score 33) |
| 900-911 | 638.9905 | 1913.9495 | 1913.9514 | 0 | R.LVMEYLPSGCLR.D |
| | | | | | 4TRAQ (N-term); compound |
| | | | | | example 1 (C); Oxidation (M) |
| | | | | | (Ions score 35) |

TABLE 16
Top 40 peaks with highest ion intensity of iTRAQ-
LVM(ox)EYLPSGC(Carbamidomethyl)LR MS/MS spectrum

| | Ion | Relative | |
|---|---|---|---|
| m/z | Intensity | Intensity | Ion |
| 115.107 | 381.8 | 4.48 | iTRAQ |
| 116.111 | 6769.6 | 79.51 | iTRAQ |
| 117.114 | 662.9 | 7.79 | iTRAQ |
| 145.109 | 3083.4 | 36.22 | |
| 175.119 | 580.7 | 6.82 | y1 |
| 230.197 | 885.3 | 10.4 | |
| 258.194 | 1633.7 | 19.19 | b1 |
| 326.961 | 424.6 | 4.99 | |
| 329.267 | 669.8 | 7.87 | |
| 340.999 | 766.1 | 9 | |
| 357.261 | 412.4 | 4.84 | |
| 402.145 | 397.1 | 4.66 | |
| 429.088 | 8513.7 | 100 | lock mass |
| 440.299 | 390.4 | 4.59 | |
| 444.331 | 564.9 | 6.64 | |
| 447.097 | 956.6 | 11.24 | |
| 569.338 | 464.6 | 5.46 | |
| 633.34 | 583.9 | 6.86 | |
| 672.315 | 1102.2 | 12.95 | |
| 673.32 | 553.5 | 6.5 | |
| 689.34 | 1439.5 | 16.91 | y6 |
| 690.341 | 1046.5 | 12.29 | |
| 802.423 | 361.6 | 4.25 | |
| 803.441 | 345.9 | 4.06 | |
| 879.49 | 331.4 | 3.89 | |
| 965.49 | 535 | 6.28 | |
| 966.489 | 780.7 | 9.17 | y8 |
| 1077.54 | 329 | 3.86 | |
| 1094.535 | 401.4 | 4.71 | y9 |
| 1095.531 | 555.8 | 6.53 | |
| 1096.533 | 392.6 | 4.61 | |
| 1177.547 | 395.8 | 4.65 | |
| 1178.572 | 1767.3 | 20.76 | y10 |
| 1234.62 | 352.4 | 4.14 | |
| 1276.628 | 424.4 | 4.98 | y11 |
| 1277.64 | 1666.4 | 19.57 | |
| 1278.624 | 563.2 | 6.62 | |
| 1389.747 | 559.1 | 6.57 | |
| 1390.721 | 1760.9 | 20.68 | [M + H − iTRAQ] |
| 1391.733 | 473.2 | 5.56 | |

TABLE 17
Top 40 peaks with highest ion intensity of iTRAQ-LVM(ox)EYLPSGC
(example 1) LR MS/MS spectrum

| | Ion | Relative | |
|---|---|---|---|
| m/z | Intensity | Intensity | Ion |
| 115.107 | 3035.7 | 58.12 | iTRAQ |
| 116.111 | 936.9 | 17.94 | iTRAQ |
| 136.076 | 5223.1 | 100 | |
| 145.107 | 1095.1 | 20.97 | |
| 175.119 | 888.5 | 17.01 | |
| 230.195 | 480.1 | 9.19 | |
| 252.65 | 372.4 | 7.13 | |
| 258.191 | 1328.4 | 25.43 | b1 |
| 285.174 | 492.4 | 9.43 | |
| 321.158 | 378.3 | 7.24 | |
| 340.987 | 334.7 | 6.41 | |
| 357.259 | 1715.5 | 32.85 | |
| 409.155 | 1177.8 | 22.55 | |
| 429.088 | 5148.8 | 98.58 | lock |
| | | | mass |
| 433.155 | 800 | 15.32 | |
| 440.297 | 838.7 | 16.06 | |
| 447.099 | 629.7 | 12.06 | |
| 503.743 | 639 | 12.23 | |
| 504.294 | 2309.4 | 44.21 | b3 |
| 505.298 | 523 | 10.01 | |
| 569.34 | 1300.4 | 24.9 | |
| 570.341 | 504.3 | 9.66 | |
| 605.345 | 742.1 | 14.21 | |
| 622.232 | 529.6 | 10.14 | |
| 633.338 | 3013.1 | 57.69 | |
| 634.341 | 1088.4 | 20.84 | |
| 691.288 | 412.3 | 7.89 | |
| 732.405 | 306.7 | 5.87 | |
| 796.41 | 433.6 | 8.3 | |
| 805.358 | 304.8 | 5.83 | |
| 822.4 | 762.3 | 14.59 | y4 |
| 909.426 | 2328.5 | 44.58 | y5 |
| 910.429 | 1009.9 | 19.33 | |
| 911.428 | 311.6 | 5.97 | |
| 989.455 | 656.6 | 12.57 | |
| 990.454 | 546.8 | 10.47 | |
| 1006.479 | 4902.3 | 93.86 | y6 |
| 1007.481 | 3551.7 | 68 | |
| 1008.481 | 642.4 | 12.3 | |
| 1119.566 | 408.2 | 7.81 | y7 |

1. A compound of formula (I) 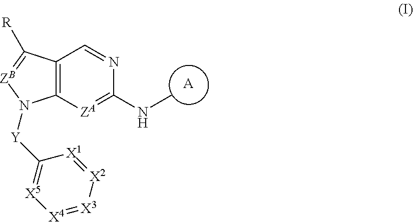
or a pharmaceutically acceptable salt thereof, wherein
R is H or F;
Z^{A} and Z^{B} are independently selected from the group consisting of CH; and N;
Ring A is phenyl, naphthyl, aromatic 5 to 6 membered heterocyclyl; or aromatic 9 to 11 membered heterobicyclyl, wherein ring A is optionally substituted with one or more R¹;
Each R¹ is independently halogen; CN; C(O)OR²; OR²; C(O)R²; C(O)N(R²R^{2a}); S(O)₂N(R²R^{2a}); S(O)N(R²R^{2a}); S(O)₂R²; S(O)R²; N(R²)S(O)₂N(R^{2a}R^{2b}); N(R²)S(O)N(R^{2a}R^{2b}); SR²; N(R²R^{2a}); NO₂; OC(O)R²; N(R²)C(O)R^{2a}; N(R²)S(O)₂R^{2a}; N(R²)S(O)R^{2a}; N(R²)C(O)N(R^{2a}R^{2b}); N(R²)C(O)OR^{2a}; OC(O)N(R²R^{2a}); T¹; C₁₋₆ alkyl; C₂₋₆ alkenyl; or C₂₋₆ alkynyl, wherein C₁₋₆ alkyl; C₂₋₆ alkenyl; and C₂₋₆ alkynyl are optionally substituted with one or more R³, which are the same or different;
R², R^{2a}, R^{2b} are independently selected from the group consisting of H; T¹; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more R³, which are the same or different;
R³ is halogen; CN; C(O)OR⁴; OR⁴; C(O)R⁴; C(O)N(R⁴R^{4a}); S(O)₂N(R⁴R^{4a}); S(O)N(R⁴R^{4a}); S(O)₂R⁴; S(O)R⁴; N(R⁴)S(O)₂N(R^{4a}R^{4b}); N(R⁴)S(O)N(R^{4a}R^{4b}); SR⁴; N(R⁴R^{4a}); NO₂; OC(O)R⁴; N(R⁴)C(O)R^{4a}; N(R⁴)S(O)₂R^{4a}; N(R⁴)S(O)R^{4a}; N(R⁴)C(O)N(R^{4a}R^{4b}); N(R⁴)C(O)OR^{4a}; OC(O)N(R⁴R^{4a}); or T¹;
R⁴, R^{4a}, R^{4b} are independently selected from the group consisting of H; T¹; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more halogen, which are the same or different;
T¹ is C₃₋₇ cycloalkyl; saturated 4 to 7 membered heterocyclyl; or saturated 7 to 11 membered heterobicyclyl, wherein T¹ is optionally substituted with one or more R¹⁰, which are the same or different;
Y is (C(R⁵R^{5a}))ₙ;
n is 0, 1, 2, 3 or 4;
R⁵, R^{5a} are independently selected from the group consisting of H; and unsubstituted C₁₋₆ alkyl; or jointly form oxo (═O);
Optionally, R⁵, R^{5a} are joined to form an unsubstituted C₃₋₇ cycloalkyl;
X¹ is C(R⁶) or N; X² is C(R^{6a}) or N; X³ is C(R^{6b}) or N; X⁴ is C(R^{6c}) or N; X⁵ is C(R^{6d}) or N, provided that at most two of X¹, X², X³, X⁴, X⁵ are N;
R⁶, R^{6a}, R^{6b}, R^{6c}, R^{6d} are independently selected from the group consisting of R^{6e}; H; halogen; CN; C(O)OR⁷; OR⁷; C(O)R⁷; C(O)N(R⁷R^{7a}); S(O)₂N(R⁷R^{7a}); S(O)N(R⁷R^{7a}); S(O)₂R⁷; S(O)R⁷; N(R⁷)S(O)₂N(R^{7a}R^{7b}); N(R⁷)S(O)N(R^{7a}R^{7b}); SR⁷; N(R⁷R^{7a}); NO₂; OC(O)R⁷; N(R⁷)C(O)R^{7a}; N(R⁷)S(O)₂R^{7a}; N(R⁷)S(O)R^{7a}; N(R⁷)C(O)N(R^{7a}R^{7b}); N(R⁷)C(O)OR^{7a}; OC(O)N(R⁷R^{7a}); T²; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more R¹¹, which are the same or different, provided that one of R⁶, R^{6a}, R^{6b}, R^{6c}, R^{6d} is R^{6e}.
R^{6e} is N(R⁷)C(O)C(R^{11a})═C(R^{11b}R^{11c}); N(R⁷)S(O)₂C(R^{11a})═C(R^{11b}R^{11c}); or N(R⁷)C(O)C≡C(R^{11a});
Optionally one of the pairs R⁶/R^{6a}, R^{6a}/R^{6b} is joined to form a ring T³;
R⁷, R^{7a}, R^{7b} are independently selected from the group consisting of H; T²; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more R⁸, which are the same or different;
R⁸ is halogen; CN; C(O)OR⁹; OR⁹; C(O)R⁹; C(O)N(R⁹R^{9a}); S(O)₂N(R⁹R^{9a}); S(O)N(R⁹R^{9a}); S(O)₂R⁹; S(O)R⁹; N(R⁹)S(O)₂N(R^{9a}R^{9b}); N(R⁹)S(O)N(R^{9a}R^{9b}); SR⁹; N(R⁹R^{9a}); NO₂; OC(O)R⁹; N(R⁹)C(O)R^{9a}; N(R⁹)S(O)₂R^{9a}; N(R⁹)S(O)R^{9a}; N(R⁹)C(O)N(R^{9a}R^{9b}); N(R⁹)C(O)OR^{9a}; OC(O)N(R⁹R^{9a}); or T²;
R⁹, R^{9a}, R^{9b} are independently selected from the group consisting of H; T²; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more R¹², which are the same or different;
R¹⁰ is halogen; CN; C(O)OR¹³; OR¹³; oxo (═O), where the ring is at least partially saturated; C(O)R¹³; C(O)N(R¹³R^{13a}); S(O)₂N(R¹³R^{13a}); S(O)N(R¹³R^{13a}); S(O)₂R¹³; S(O)R¹³; N(R¹³)S(O)₂N(R^{13a}R^{13b}); N(R¹³)S(O)N(R^{13a}R^{13b}); SR¹³; N(R¹³R^{13a}); NO₂; OC(O)R¹³; N(R¹³)C(O)R^{13a}; N(R¹³)S(O)₂R^{13a}; N(R¹³)S(O)R^{13a}; N(R¹³)C(O)N(R^{13a}R^{13b}); N(R¹³)C(O)OR^{13a}; OC(O)N(R¹³R^{13a}); C₁₋₆ alkyl; C₂₋₆ alkenyl; or C₂₋₆ alkynyl, wherein C₁₋₆ alkyl; C₂₋₆ alkenyl; and C₂₋₆ alkynyl are optionally substituted with one or more R¹⁴, which are the same or different;
R¹³, R^{13a}, R^{13b} are independently selected from the group consisting of H; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more R¹⁴, which are the same or different;
R¹¹, R¹² are independently selected from the group consisting of halogen; CN; C(O)OR¹⁵; OR¹⁵; C(O)R¹⁵; C(O)N(R¹⁵R^{15a}); S(O)₂N(R¹⁵R^{15a}); S(O)N(R¹⁵R^{15a}); S(O)₂R¹⁵; S(O)R¹⁵; N(R¹⁵)S(O)₂N(R^{15a}R^{15b}); N(R¹⁵)S(O)N(R^{15a}R^{15b}); SR¹⁵; N(R¹⁵R^{15a}); NO₂; OC(O)R¹⁵; N(R¹⁵)C(O)R^{15a}; N(R¹⁵)S(O)₂R^{15a}; N(R¹⁵)S(O)R^{15a}; N(R¹⁵)C(O)N(R^{15a}R^{15b}); N(R¹⁵)C(O)OR^{15a}; OC(O)N(R¹⁵R^{15a}); and T²; R^{11a}, R^{11b}, R^{11c} are independently selected from the group consisting of H; halogen; CN; OR¹⁵; C(O)N(R¹⁵R^{15a}); and C₁₋₆ alkyl, wherein C₁₋₆ alkyl is optionally substituted with one or more R¹⁴, which are the same or different;
R¹⁵, R^{15a}, R^{15b} are independently selected from the group consisting of H; T²; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more halogen, which are the same or different;
R¹⁴ is halogen; CN; C(O)OR¹⁶; OR¹⁶; C(O)R¹⁶; C(O)N(R¹⁶R^{16a}); S(O)₂N(R¹⁶R^{16a}); S(O)N(R¹⁶R^{16a}); S(O)₂R¹⁶; S(O)R¹⁶; N(R¹⁶)S(O)₂N(R^{16a}R¹⁶); N(R¹⁶)S(O)N(R^{16a}R^{16b}); SR¹⁶; N(R¹⁶R^{16a}); NO₂; OC(O)R¹⁶; N(R¹⁶)C(O)R^{16a}; N(R¹⁶)S(O)₂R^{16a}; N(R¹⁶)S(O)R^{16a}; N(R¹⁶)C(O)N(R^{16a}R^{16b}); N(R¹⁶)C(O)OR^{16a}; or OC(O)N(R¹⁶R^{16a});
R¹⁶, R^{16a}, R^{16b} are independently selected from the group consisting of H; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more halogen, which are the same or different;
T² is phenyl; naphthyl; indenyl; indanyl; C₃₋₇ cycloalkyl; 4 to 7 membered heterocyclyl; or 7 to 11 membered heterobicyclyl, wherein T² is optionally substituted with one or more R¹⁷, which are the same or different;
T³ is phenyl; C₃₋₇ cycloalkyl; or 4 to 7 membered heterocyclyl, wherein T³ is optionally substituted with one or more R¹⁸, which are the same or different;
R¹⁷, R¹⁸ are independently selected from the group consisting of halogen; CN; C(O)OR¹⁹; OR¹⁹; oxo (═O), where the ring is at least partially saturated; C(O)R¹⁹; C(O)N(R¹⁹R^{19a}); S(O)₂N(R¹⁹R^{19a}); S(O)N(R¹⁹R^{19a}); S(O)₂R¹⁹; S(O)R¹⁹; N(R¹⁹)S(O)₂N(R^{19a}R^{19b}); N(R¹⁹)S(O)N(R^{19a}R^{19b}); SR¹⁹, N(R¹⁹R^{19a}); NO₂; OC(O)R¹⁹; N(R¹⁹)C(O)R^{19a}; N(R¹⁹)S(O)₂R^{19a}; N(R¹⁹)S(O)R^{19a}; N(R¹⁹)C(O)N(R^{19a}R^{19b}); N(R¹⁹)C(O)OR^{19a}; OC(O)N(R¹⁹R^{19a}); C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more R²⁰, which are the same or different;
R¹⁹, R^{19a}, R^{19b} are independently selected from the group consisting of H; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more R²⁰, which are the same or different;
R²⁰ is halogen; CN; C(O)OR²¹; OR²¹; C(O)R²¹; C(O)N(R²¹R^{21a}); S(O)₂N(R²¹R^{21a}); S(O)N(R²¹R^{21a}); S(O)₂R²¹; S(O)R²¹; N(R²¹)S(O)₂N(R^{21a}R^{21b}); NR²¹)S(O)N(R^{21a}R^{21b}); SR²¹; N(R²¹R^{21a}); NO₂; OC(O)R²¹; N(R²¹)C(O)R^{21a}; N(R²¹)S(O)₂R^{21a}; N(R²¹)S(O)R^{21a}; N(R²¹)C(O)N(R^{21a}R^{21b}) N(R²¹)C(O)OR^{21a}; or OC(O)N(R²¹R^{21a});
R²¹, R^{21a}, R^{21b} are independently selected from the group consisting of H; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more halogen, which are the same or different.

2. The compound of claim 1, wherein ring A, Z^{A}, Z^{B} in formula (I) are defined to give formula (Ia) 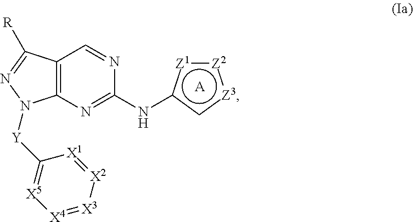
wherein ring A is a aromatic 5 membered heterocycle in which Z¹, Z² and Z³ are independently selected from the group consisting of C(R¹), N, N(R¹), O and S, provided that at least one of Z¹, Z², Z³ is N; and wherein R, Y, X¹ to X⁵ and R¹ are defined as in claim 1.

3. The compound of claim 1, wherein ring A, Z^{A}, Z^{B}, R in formula (I) are defined to give formula (Ib) 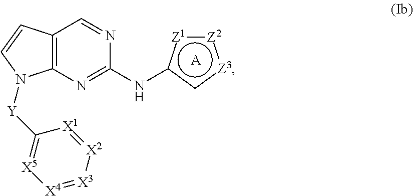
wherein ring A is a membered 5 aromatic heterocycle in which Z¹, Z² and Z³ are independently selected from the group consisting of C(R¹), N, N(R¹), O and S, provided that at least one of Z¹, Z², Z³ is N; and wherein Y, X¹ to X⁵ and R¹ are defined as in claim 1.

4. The compound of claim 1, wherein ring A, Z^{A}, Z^{B} in formula (I) are defined to give formula (Ic) 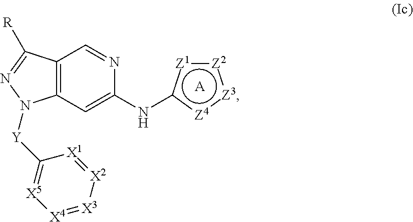
wherein ring A is a aromatic 5 membered heterocycle in which Z¹, Z², Z³ and Z⁴ are independently selected from the group consisting of C(R¹), N, N(R¹), O and S, provided that at least one of Z¹, Z², Z³, Z⁴ is N or N(R¹); and wherein R, Y, X¹ to X⁵ and R¹ are defined as in claim 1.

5. The compound of claim 1, wherein A, Z^{A}, Z^{B} in formula (I) are defined to give formula (Id) 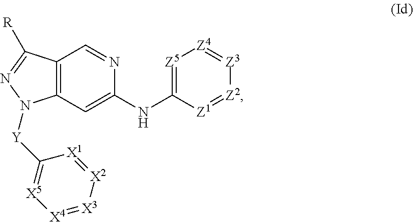
wherein in ring A Z¹ is C(R¹) or N; Z² is C(R¹) or N; Z³ is C(R¹) or N; Z⁴ is C(R¹) or N; Z⁵ is C(R¹), or N, provided that at most two of Z¹, Z², Z³, Z⁴, Z⁵ are N;
optionally two adjacent R¹ are joined to form together with the ring including Z¹ to Z⁵ an aromatic bicyclic ring T⁰;
T⁰ is aromatic 9 to 11 membered heterobicyclyl; naphthyl; indenyl; or indanyl, wherein T⁰ is optionally substituted with one or more R^{1a}, which are the same or different;
R^{1a} is halogen; CN; C(O)OR²; OR²; oxo (═O), where the ring is at least partially saturated; C(O)R²; C(O)N(R²R^{2a}); S(O)₂N(R²R^{2a}); S(O)N(R²R^{2a}); S(O)₂R²; S(O)R²; N(R²)S(O)₂N(R^{2a}R^{2b}); N(R²)S(O)N(R^{2a}R^{2b}); SR²; N(R²R^{2a}); NO₂; OC(O)R²; N(R²)C(O)R^{2a}; N(R²)S(O)₂R^{2a}; N(R²)S(O)R^{2a}; N(R²)C(O)N(R^{2a}R^{2b}); N(R²)C(O)OR^{2a}; OC(O)N(R²R^{2a}); T¹; or C₁₋₆ alkyl, wherein C₁ alkyl is optionally substituted with one or more R³, which are the same or different; and
wherein R, R¹, R², R^{2a}; R^{2b}, R³; Y, X¹ to X⁵ and R¹ are defined as in claim 1.

6. The compound of any one of claims 1 to 5, wherein R is H.

7. The compound of any one of claims 1 to 6, wherein Y is CH₂.

8. The compound of any one of claims 1 to 7, wherein none or one of R⁶, R^{6a}, R^{6b}, R^{6c}, R^{6d} is N.

9. The compound of any one of claims 1 to 8, wherein R⁶, R^{6a}, R^{6b}, R^{6c}, R^{6d} are independently selected from the group consisting of R^{6e}; H; halogen; and C₁₋₆ alkyl, wherein C₁₋₆ alkyl is optionally substituted with one or more halogen, which are the same or different, provided that one of R⁶, R^{6a}, R^{6b}, R^{6e}, R^{6d} is R^{6e}.

10. The compound of any one of claims 1 to 9, wherein R⁷, R^{11a}, R^{11b}, R^{11c} are independently selected from the group consisting of H; and C₁₋₄ alkyl, wherein C₁₋₄ alkyl is optionally substituted with one or more halogen, which are the same or different.

11. The compound of any one of claims 1 to 10, wherein R^{6a} is R^{6e}.

12. The compound of any one of claims 1 to 11, wherein R^{6e} is NHC(O)CH═CH₂; NHC(O)C(CH₃)═CH₂; NHC(O)CH═C(CH₃)₂; NHS(O)₂CH═CH₂; or NHC(O)C≡CH.

13. The compound of any one of claims 1 to 12, wherein ring A is a pyrazole, an oxazole, an isoxazole, a triazole, a phenyl, or a pyridyl ring.

14. The compound of any one of claims 1 to 13, wherein 0, or 2 R¹, which are the same or different, are other than H.

15. The compound of any one of claims 1 to 14, wherein R¹ is C₁₋₄ alkyl, which is optionally substituted with 1 or 2 R³, which are the same or different.

16. The compound of any one of claims 1 to 15, wherein R³ is halogen; CN; OR⁴; C(O)N(R⁴R^{4a}); or C(O)T¹.

17. The compound of any one of claims 1 to 16 or a pharmaceutically acceptable salt thereof selected from the group consisting of
N-(3-((6-((1-Methyl-1H-pyrazol-4-yl)amino)-1H-pyrazolo[3,4-d]pyrimidin-1-yl)methyl)phenyl)acrylamide;
N-(2-Fluoro-5-((6-((1-methyl-1H-pyrazol-4-yl)amino)-1H-pyrazolo[3,4-d]pyrimidin-1-yl)methyl)phenyl)acrylamide;
N-(3-((6-((1-Methyl-1H-pyrazol-4-yl)amino)-1H-pyrazolo[3,4-d]pyrimidin-1-yl)methyl)phenyl)propiolamide;
N-(3-((6-((1-Methyl-1H-pyrazol-4-yl)amino)-1H-pyrazolo[3,4-d]pyrimidin-1-yl)methyl)phenyl)ethenesulfonamide;
N-(3-((6-((1-Methyl-1H-pyrazol-4-yl)amino)-1H-pyrazolo[3,4-d]pyrimidin-1-yl)methyl)phenyl)methacrylamide;
3-Methyl-N-(3-((6-((1-methyl-1H-pyrazol-4-yl)amino)-1H-pyrazolo[3,4-d]pyrimidin-1-yl)methyl)phenyl)but-2-enamide;
N-(3-((2-((1-Methyl-1H-pyrazol-4-yl)amino)-7H-pyrrolo[2,3-d]pyrimidin-7-yl)methyl)phenyl)ethenesulfonamide; and
N-(3-((6-((1-Methyl-1H-pyrazol-3-yl)amino)-1H-pyrazolo[4,3-c]pyridin-1-yl)methyl)phenyl)acrylamide;
N-(3-((2-((1-Methyl-1H-pyrazol-4-yl)amino)-7H-pyrrolo[2,3-d]pyrimidin-7-yl)methyl)phenyl)acrylamide; and
N-(3-((6-((1-Methyl-1H-pyrazol-4-yl)amino)-1H-pyrazolo[4,3-c]pyridin-1-yl)methyl)phenyl)acrylamide.

18. A pharmaceutical composition comprising a compound or a pharmaceutically acceptable salt thereof of any claims 1 to 17 together with a pharmaceutically acceptable carrier, optionally in combination with one or more other pharmaceutical compositions.

19. A compound or a pharmaceutically acceptable salt thereof of any claims 1 to 17 for use as a medicament.

20. A compound or a pharmaceutically acceptable salt thereof of any claims 1 to 17 for use in a method of treating or preventing a disease or disorder associated with JAK3, BTK, BLK, ITK or TEC.

21. A compound or a pharmaceutically acceptable salt thereof of any one of claims 1 to 17 for use in a method of treating or preventing an immunological, inflammatory, autoimmune, or allergic disorder or disease of a transplant rejection or a Graft-versus host disease.

22. A compound or a pharmaceutically acceptable salt thereof of any one of claims 1 to 17 for use in a method of treating or preventing a proliferative disease.

23. Use of a compound of any one of claims 1 to 17 or a pharmaceutically acceptable salt thereof for the manufacture of a medicament for the treatment or prophylaxis of diseases and disorders associated with JAK3, BTK, BLK, ITK or TEC.

24. A method for treating, controlling, delaying or preventing in a mammalian patient in need thereof one or more conditions selected from the group consisting of diseases and disorders associated with JAK3, BTK, BLK, ITK or TEC, wherein the method comprises the administration to said patient a therapeutically effective amount of a compound of any one of claims 1 to 17 or a pharmaceutically acceptable salt thereof.


SEQUENCE LISTING (1 sequence)

SEQ ID NO: 1 - protein, 1124 aa, Homo sapiens
MAPPSEETPLIPQRSCSLLSTEAGALHVLLPARGPGPPQRLSFSFGDHLAEDLCVQAAKASGILPVYHSLFALATEDLSCWFPPSHIFSVEDASTQVLLYRIRFYFPNWFGLEKCHRFGLRKDLASAILDLPVLEHLFAQHRSDLVSGRLPVGLSLKEQGECLSLAVLDLARMAREQAQRPGELLKTVSYKACLPPSLRDLIQGLSFVTRRRIRRTVRRALRRVAACQADRHSLMAKYIMDLERLDPAGAAETFHVGLPGALGGHDGLGLLRVAGDGGIAWTQGEQEVLQPFCDFPEIVDISIKQAPRVGPAGEHRLVTVTRTDNQILEAEFPGLPEALSFVALVDGYFRLTTDSQHFFCKEVAPPRLLEEVAEQCHGPITLDFAINKLKTGGSRPGSYVLRRSPQDFDSFLLTVCVQNPLGPDYKGCLIRRSPTGTFLLVGLSRPHSSLRELLATCWDGGLHVDGVAVTLTSCCIPRPKEKSNLIVVQRGHSPPTSSLVQPQSQYQLSQMTFHKIPADSLEWHENLGHGSFTKIYRGCRHEVVDGEARKTEVLLKVMDAKHKNCMESFLEAASLMSQVSYRHLVLLHGVCMAGDSTMVQEFVHLGAIDMYLRKRGHLVPASWKLQVVKQLAYALNYLEDKGLPHGNVSARKVLLAREGADGSPPFIKLSDPGVSPAVLSLEMLTDRIPWVAPECLREAQTLSLEADKWGFGATVWEVFSGVTMPISALDPAKKLQFYEDRQQLPAPKWTELALLIQQCMAYEPVQRPSFRAVIRDLNSLISSDYELLSDPTPGALAPRDGLWNGAQLYACQDPTIFEERHLKYISQLGKGNFGSVELCRYDPLGDNTGALVAVKQLQHSGPDQQRDFQREIQILKALHSDFIVKYRGVSYGPGRQSLRLVMEYLPSGCLRDFLQRHRARLDASRLLLYSSQICKGMEYLGSRRCVHRDLAARNILVESEAHVKIADFGLAKLLPLDKDYYVVREPGQSPIFWYAPESLSDNIFSRQSDVWSFGVVLYELFTYCDKSCSPSAEFLRMMGCERDVPALCRLLELLEEGQRLPAPPACPAEVHELMKLCWAPSPQDRPSFSALGPQLDMLWSGSRGCETHAFTAHPEGKHHSLSFS